SEQUENCE LISTING (101 sequences)

SEQ ID NO: 1 - DNA, 912 bp, Homo sapiens
atgcccgaggagtcctcgccccgccgcactccccagtctattccataccaggatctcccccacctggtgaacgctgacggtcagtacctcttctgcaggtactggaagcccaccggcacgccaaaggcgctgatcttcgtctcccacggggccggggaacactccggccgctacgaggagttagccaggatgctgatgggcctggacctgctggtatttgcccacgaccacgtgggccacggacaatcagagggcgagagaatggtggtttccgatttccacgtgttcgtgagggacgtcctccagcacgtggactccatgcagaaagactacccagggctacccgtctttctgctcgggcattccatggggggggcaatcgccatcctgaccgccgccgagcgtcctggccacttcgccgggatggtcctgatttcacccctcgtgctggccaaccctgagtctgcgacaaccttcaaggtgctggcggccaaggtactcaacctggtgctgcccaacctgtcgctcggccctatcgactcctcagtgctctccaggaacaagacagaggttgacatctacaacagcgatcccctgatctgtcgcgccggcctcaaggtatgcttcggaatccagctgctgaacgccgtctcccgcgttgagcgcgccttacccaagcttacggtgccttttctgttgctgcagggctccgccgatcggctgtgcgattccaagggcgcttacctgctgatggaactagctaagtcccaggataagaccctgaagatctacgaaggggcctaccacgtcctgcacaaggagctgcccgaagtgaccaactccgtgttccacgagatcaacatgtgggtgagtcagcggacagcgaccgccgggaccgcatctccgccttga

SEQ ID NO: 2 - DNA, 912 bp, Homo sapiens
atgccagaggaaagttcccccaggcggaccccgcagagcattccctaccaggacctccctcacctggtcaatgcagacggacagtacctcttctgcaggtactggaaacccacaggcacacccaaggccctcatctttgtgtcccatggagccggagagcacagtggccgctatgaagagctggctcggatgctgatggggctggacctgctggtgttcgcccacgaccatgttggccacggacagagcgaaggggagaggatggtagtgtctgacttccacgttttcgtcagggatgtgttgcagcatgtggattccatgcagaaagactaccctgggcttcctgtcttccttctgggccactccatgggaggcgccatcgccatcctcacggccgcagagaggccgggccacttcgccggcatggtactcatttcgcctctggttcttgccaatcctgaatctgcaacaactttcaaggtccttgctgcgaaagtgctcaaccttgtgctgccaaacttgtccctcgggcccatcgactccagcgtgctctctcggaataagacagaggtcgacatttataactcagaccccctgatctgccgggcagggctgaaggtgtgcttcggcatccaactgctgaatgccgtctcacgggtggagcgcgccctccccaagctgactgtgcccttcctgctgctccagggctctgccgatcgcctatgtgacagcaaaggggcctacctgctcatggagttagccaagagccaggacaagactctcaagatttatgaaggtgcctaccatgttctccacaaggagcttcctgaagtcaccaactccgtcttccatgaaataaacatgtgggtctctcaaaggacagccacggcaggaactgcgtccccaccctga

SEQ ID NO: 3 - DNA, 20 bp, Homo sapiens
gagggcatatgatgccagaa

SEQ ID NO: 4 - DNA, 40 bp, Homo sapiens
gagagctccccccgcagaaccccccagagtattccttatc

SEQ ID NO: 5 - DNA, 40 bp, Homo sapiens
aggatctgcctcacctggtgaacgcagatggtcagtattt

SEQ ID NO: 6 - DNA, 40 bp, Homo sapiens
attctgtcggtactggaaacctacggggaccccgaaagca

SEQ ID NO: 7 - DNA, 40 bp, Homo sapiens
cttatctttgtcagccatggtgcgggggagcactccggtc

SEQ ID NO: 8 - DNA, 40 bp, Homo sapiens
gatacgaagaactggctcgcatgcttatgggcctggatct

SEQ ID NO: 9 - DNA, 40 bp, Homo sapiens
gctggtgtttgcacatgatcatgtcggccacggtcagtcg

SEQ ID NO: 10 - DNA, 40 bp, Homo sapiens
gaaggtgaacgcatggtagtgtcggattttcatgtgtttg

SEQ ID NO: 11 - DNA, 40 bp, Homo sapiens
tacgtgacgtgttgcaacatgttgatagcatgcagaaaga

SEQ ID NO: 12 - DNA, 40 bp, Homo sapiens
ctatccaggtctccctgtattcctgcttgggcatagcatg

SEQ ID NO: 13 - DNA, 40 bp, Homo sapiens
gggggtgctatcgcgattctgaccgcggcggagcgcccgg

SEQ ID NO: 14 - DNA, 40 bp, Homo sapiens
gccactttgcaggcatggtactgatctcgccgctggtgtt

SEQ ID NO: 15 - DNA, 40 bp, Homo sapiens
ggcgaatccggaatccgccaccaccttcaaggtgctggct

SEQ ID NO: 16 - DNA, 40 bp, Homo sapiens
gcaaaagtgctgaacctggttctccctaatttgagcctgg

SEQ ID NO: 17 - DNA, 40 bp, Homo sapiens
gccccattgacagcagcgtactctcaaggaataaaaccga

SEQ ID NO: 18 - DNA, 40 bp, Homo sapiens
ggtggatatttacaattcagatccgctgatatgtcgtgcc

SEQ ID NO: 19 - DNA, 40 bp, Homo sapiens
ggccttaaagtatgtttcggcattcagctgctgaacgcgg

SEQ ID NO: 20 - DNA, 40 bp, Homo sapiens
tgtcgcgcgtagagcgggcgctgcctaaactgacagtgcc

SEQ ID NO: 21 - DNA, 40 bp, Homo sapiens
cttcctgctgctgcagggcagtgccgaccgcctgtgtgat

SEQ ID NO: 22 - DNA, 40 bp, Homo sapiens
tcgaaaggcgcgtatctgcttatggagctcgcaaaatccc

SEQ ID NO: 23 - DNA, 40 bp, Homo sapiens
aagacaaaactctgaaaatatatgagggtgcctatcatgt

SEQ ID NO: 24 - DNA, 40 bp, Homo sapiens
tctccacaaagaactgcctgaggtcaccaattcagttttc

SEQ ID NO: 25 - DNA, 40 bp, Homo sapiens
cacgaaataaatatgtgggtttcccagcgcaccgctactg

SEQ ID NO: 26 - DNA, 40 bp, Homo sapiens
caggcaccgccagccccccgtgatctagactcgagaatac

SEQ ID NO: 27 - DNA, 20 bp, Homo sapiens
gtattctcgagtctagatca

SEQ ID NO: 28 - DNA, 40 bp, Homo sapiens
cggggggctggcggtgcctgcagtagcggtgcgctgggaa

SEQ ID NO: 29 - DNA, 40 bp, Homo sapiens
acccacatatttatttcgtggaaaactgaattggtgacct

SEQ ID NO: 30 - DNA, 40 bp, Homo sapiens
caggcagttctttgtggagaacatgataggcaccctcata

SEQ ID NO: 31 - DNA, 40 bp, Homo sapiens
tattttcagagttttgtcttgggattttgcgagctccata

SEQ ID NO: 32 - DNA, 40 bp, Homo sapiens
agcagatacgcgcctttcgaatcacacaggcggtcggcac

SEQ ID NO: 33 - DNA, 40 bp, Homo sapiens
tgccctgcagcagcaggaagggcactgtcagtttaggcag

SEQ ID NO: 34 - DNA, 40 bp, Homo sapiens
cgcccgctctacgcgcgacaccgcgttcagcagctgaatg

SEQ ID NO: 35 - DNA, 40 bp, Homo sapiens
ccgaaacatactttaaggccggcacgacatatcagcggat

SEQ ID NO: 36 - DNA, 40 bp, Homo sapiens
ctgaattgtaaatatccacctcggttttattccttgagag

SEQ ID NO: 37 - DNA, 40 bp, Homo sapiens
tacgctgctgtcaatggggcccaggctcaaattagggaga

SEQ ID NO: 38 - DNA, 40 bp, Homo sapiens
accaggttcagcacttttgcagccagcaccttgaaggtgg

SEQ ID NO: 39 - DNA, 40 bp, Homo sapiens
tggcggattccggattcgccaacaccagcggcgagatcag

SEQ ID NO: 40 - DNA, 40 bp, Homo sapiens
taccatgcctgcaaagtggcccgggcgctccgccgcggtc

SEQ ID NO: 41 - DNA, 40 bp, Homo sapiens
agaatcgcgatagcaccccccatgctatgcccaagcagga

SEQ ID NO: 42 - DNA, 40 bp, Homo sapiens
atacagggagacctggatagtctttctgcatgctatcaac

SEQ ID NO: 43 - DNA, 40 bp, Homo sapiens
atgttgcaacacgtcacgtacaaacacatgaaaatccgac

SEQ ID NO: 44 - DNA, 40 bp, Homo sapiens
actaccatgcgttcaccttccgactgaccgtggccgacat

SEQ ID NO: 45 - DNA, 40 bp, Homo sapiens
gatcatgtgcaaacaccagcagatccaggcccataagcat

SEQ ID NO: 46 - DNA, 40 bp, Homo sapiens
gcgagccagttcttcgtatcgaccggagtgctcccccgca

SEQ ID NO: 47 - DNA, 40 bp, Homo sapiens
ccatggctgacaaagataagtgctttcggggtccccgtag

SEQ ID NO: 48 - DNA, 40 bp, Homo sapiens
gtttccagtaccgacagaataaatactgaccatctgcgtt

SEQ ID NO: 49 - DNA, 40 bp, Homo sapiens
caccaggtgaggcagatcctgataaggaatactctggggg

SEQ ID NO: 50 - DNA, 40 bp, Homo sapiens
gttctgcggggggagctctcttctggcatcatatgccctc

SEQ ID NO: 51 - DNA, 20 bp, Homo sapiens
ttcaactcatatgcccgagg

SEQ ID NO: 52 - DNA, 40 bp, Homo sapiens
agtcctcgccccgccgcactccccagtctattccatacca

SEQ ID NO: 53 - DNA, 40 bp, Homo sapiens
ggatctcccccacctggtgaacgctgacggtcagtacctc

SEQ ID NO: 54 - DNA, 40 bp, Homo sapiens
ttctgcaggtactggaagcccaccggcacgccaaaggcgc

SEQ ID NO: 55 - DNA, 40 bp, Homo sapiens
tgatcttcgtctcccacggggccggggaacactccggccg

SEQ ID NO: 56 - DNA, 40 bp, Homo sapiens
ctacgaggagttagccaggatgctgatgggcctggacctg

SEQ ID NO: 57 - DNA, 40 bp, Homo sapiens
ctggtatttgcccacgaccacgtgggccacggacaatcag

SEQ ID NO: 58 - DNA, 40 bp, Homo sapiens
agggcgagagaatggtggtttccgatttccacgtgttcgt

SEQ ID NO: 59 - DNA, 40 bp, Homo sapiens
gagggacgtcctccagcacgtggactccatgcagaaagac

SEQ ID NO: 60 - DNA, 40 bp, Homo sapiens
tacccagggctacccgtctttctgctcgggcattccatgg

SEQ ID NO: 61 - DNA, 40 bp, Homo sapiens
ggggggcaatcgccatcctgaccgccgccgagcgtcctgg

SEQ ID NO: 62 - DNA, 40 bp, Homo sapiens
ccacttcgccgggatggtcctgatttcacccctcgtgctg

SEQ ID NO: 63 - DNA, 40 bp, Homo sapiens
gccaaccctgagtctgcgacaaccttcaaggtgctggcgg

SEQ ID NO: 64 - DNA, 40 bp, Homo sapiens
ccaaggtactcaacctggtgctgcccaacctgtcgctcgg

SEQ ID NO: 65 - DNA, 40 bp, Homo sapiens
ccctatcgactcctcagtgctctccaggaacaagacagag

SEQ ID NO: 66 - DNA, 40 bp, Homo sapiens
gttgacatctacaacagcgatcccctgatctgtcgcgccg

SEQ ID NO: 67 - DNA, 40 bp, Homo sapiens
gcctcaaggtatgcttcggaatccagctgctgaacgccgt

SEQ ID NO: 68 - DNA, 40 bp, Homo sapiens
ctcccgcgttgagcgcgccttacccaagcttacggtgcct

SEQ ID NO: 69 - DNA, 40 bp, Homo sapiens
tttctgttgctgcagggctccgccgatcggctgtgcgatt

SEQ ID NO: 70 - DNA, 40 bp, Homo sapiens
ccaagggcgcttacctgctgatggaactagctaagtccca

SEQ ID NO: 71 - DNA, 40 bp, Homo sapiens
ggataagaccctgaagatctacgaaggggcctaccacgtc

SEQ ID NO: 72 - DNA, 40 bp, Homo sapiens
ctgcacaaggagctgcccgaagtgaccaactccgtgttcc

SEQ ID NO: 73 - DNA, 40 bp, Homo sapiens
acgagatcaacatgtgggtgagtcagcggacagcgaccgc

SEQ ID NO: 74 - DNA, 40 bp, Homo sapiens
cgggaccgcatctccgccttgatctagactcgaggaccct

SEQ ID NO: 75 - DNA, 20 bp, Homo sapiens
agggtcctcgagtctagatc

SEQ ID NO: 76 - DNA, 40 bp, Homo sapiens
aaggcggagatgcggtcccggcggtcgctgtccgctgact

SEQ ID NO: 77 - DNA, 40 bp, Homo sapiens
cacccacatgttgatctcgtggaacacggagttggtcact

SEQ ID NO: 78 - DNA, 40 bp, Homo sapiens
tcgggcagctccttgtgcaggacgtggtaggccccttcgt

SEQ ID NO: 79 - DNA, 40 bp, Homo sapiens
agatcttcagggtcttatcctgggacttagctagttccat

SEQ ID NO: 80 - DNA, 40 bp, Homo sapiens
cagcaggtaagcgcccttggaatcgcacagccgatcggcg

SEQ ID NO: 81 - DNA, 40 bp, Homo sapiens
gagccctgcagcaacagaaaaggcaccgtaagcttgggta

SEQ ID NO: 82 - DNA, 40 bp, Homo sapiens
aggcgcgctcaacgcgggagacggcgttcagcagctggat

SEQ ID NO: 83 - DNA, 40 bp, Homo sapiens
tccgaagcataccttgaggccggcgcgacagatcagggga

SEQ ID NO: 84 - DNA, 40 bp, Homo sapiens
tcgctgttgtagatgtcaacctctgtcttgttcctggaga

SEQ ID NO: 85 - DNA, 40 bp, Homo sapiens
gcactgaggagtcgatagggccgagcgacaggttgggcag

SEQ ID NO: 86 - DNA, 40 bp, Homo sapiens
caccaggttgagtaccttggccgccagcaccttgaaggtt

SEQ ID NO: 87 - DNA, 40 bp, Homo sapiens
gtcgcagactcagggttggccagcacgaggggtgaaatca

SEQ ID NO: 88 - DNA, 40 bp, Homo sapiens
ggaccatcccggcgaagtggccaggacgctcggcggcggt

SEQ ID NO: 89 - DNA, 40 bp, Homo sapiens
caggatggcgattgccccccccatggaatgcccgagcaga

SEQ ID NO: 90 - DNA, 40 bp, Homo sapiens
aagacgggtagccctgggtagtctttctgcatggagtcca

SEQ ID NO: 91 - DNA, 40 bp, Homo sapiens
cgtgctggaggacgtccctcacgaacacgtggaaatcgga

SEQ ID NO: 92 - DNA, 40 bp, Homo sapiens
aaccaccattctctcgccctctgattgtccgtggcccacg

SEQ ID NO: 93 - DNA, 40 bp, Homo sapiens
tggtcgtgggcaaataccagcaggtccaggcccatcagca

SEQ ID NO: 94 - DNA, 40 bp, Homo sapiens
tcctggctaactcctcgtagcggccggagtgttccccggc

SEQ ID NO: 95 - DNA, 40 bp, Homo sapiens
cccgtgggagacgaagatcagcgcctttggcgtgccggtg

SEQ ID NO: 96 - DNA, 40 bp, Homo sapiens
ggcttccagtacctgcagaagaggtactgaccgtcagcgt

SEQ ID NO: 97 - DNA, 40 bp, Homo sapiens
tcaccaggtgggggagatcctggtatggaatagactgggg

SEQ ID NO: 98 - DNA, 40 bp, Homo sapiens
agtgcggcggggcgaggactcctcgggcatatgagttgaa

SEQ ID NO: 99 - protein, 303 aa, Homo sapiens
MPEESSPRRTPQSIPYQDLPHLVNADGQYLFCRYWKPTGTPKALIFVSHGAGEHSGRYEELARMLMGLDLLVFAHDHVGHGQSEGERMVVSDFHVFVRDVLQHVDSMQKDYPGLPVFLLGHSMGGAIAILTAAERPGHFAGMVLISPLVLANPESATTFKVLAAKVLNLVLPNLSLGPIDSSVLSRNKTEVDIYNSDPLICRAGLKVCFGIQLLNAVSRVERALPKLTVPFLLLQGSADRLCDSKGAYLLMELAKSQDKTLKIYEGAYHVLHKELPEVTNSVFHEINMWVSQRTATAGTASPP

SEQ ID NO: 100 - DNA, 15 bp, Escherichia coli
atgaatcacaaagtg

SEQ ID NO: 101 - protein, 5 aa, Escherichia coli
MNHKV

(19) United States
(12) Patent Application Publication
(10) Pub. No.: US 2009/0311723 A1
(43) Pub. Date: Dec. 17, 2009

(54) FLUORESCENCE-BASED ASSAY FOR MONOACYLGLYCEROL LIPASE COMPATIBLE WITH INHIBITOR SCREENING

(75) Inventors: Pranab Kumar Chanda, West Windsor, NJ (US); Yuren Wang, Plainsboro, NJ (US)

Correspondence Address: WYETH;PATENT LAW GROUP, 5 GIRALDA FARMS, MADISON, NJ, 07940, US

(73) Assignee: WYETH, Madison, NJ (US)

(21) Appl. No.: 12/275,343

(22) Filed: Nov. 21, 2008

Related U.S. Application Data
(60) Provisional application No. 60/989,598, filed on Nov. 21, 2007.

(51) Int. Cl. G01N 33/53; C07H 21/04; C12N 9/14

(52) U.S. Cl. 435/7.72; 536/23.5; 435/195

(57) ABSTRACT

The present invention provides reagents, kits and methods for assaying monoglycerol lipase activity and for identifying compounds that modulate monoglycerol lipase (“MGL”) activity. A simple, sensitive fluorescence assay, which is amenable to high throughput screening, is described. In one embodiment, 7-Hydroxycoumarinyl-arachidonate (7-HCA) is used as a fluorogenic substrate for MGL, which catalyzes the hydrolysis of 7-HCA to generate arachidonic acid and the highly fluorescent 7-hydroxycoumarin (7-HC). Release of 7-HC is monitored continuously using a fluorometer. MGL protein catalyzed the hydrolysis of 7-HCA with an apparent KM of 9.8 mM and Vmax of 1.7 mmoles min⁻¹ mg protein⁻¹.

### FIELD

The invention relates generally to reagents, kits and methods for detecting lipase activity; more specifically to reagents, kits and methods for identifying compounds that modulate monoglycerol lipase activity and have therapeutic potential for modulating endocannabinoid metabolism.

### BACKGROUND

The management of pain is an important medical and economic endeavor. Current treatments for pain include the administration of non-steroidal anti-inflammatory drugs (“NSAIDS”), acetaminophen, and opioids, all of which have limitations. Notably, morphine-based drugs are generally regarded to be ineffective at treating hyperalgesia, allodynia and neuropathic pain. Thus, the identification of new classes of antinociceptives addresses a long felt medical need.

The endogenous cannabinoid system has been postulated to affect the nociception (pain sensing) pathway at various levels (see Iverson and Chapmen, Curr. Opin. Pharmacol. 2002, 2:50-55, which is incorporated herein by reference). At least, two types of cannabinoid receptors have been identified, CB₁ (also called CNR1) and CB₂ (also called CNR2). The CB₁ receptors function in the central nervous system as well as the peripheral nervous system, whereas the CB₂ receptors are more or less restricted to the peripheral nervous system with some CNS expression. The cannabinoid system is also implicated in obesity, dyslipidemia, alcoholism and other addictions, inflammation, and allergies, and is therefore an important pharmacological target.

Multiple endocannabinoid-like (“ECL”) molecules have been identified (reviewed in Alexander and Kendall, Brit. J. Pharmacol. (2007), 1-22, which is incorporated herein by reference). The two major classes of ECL molecules are N-linked and O-linked molecules. One well-studied N-linked ECL is anandamide (also known as acylethanolamine or AEA). Anandamide is thought to be formed in vivo primarily via hydrolysis of the membrane phospholipid N-acylphosphatidyl-ethanolamine (NAPE) by the enzyme N-acylphosphatidyl-ethanolamine phospholipase D (NAPE-PLD). An O-linked ECL, 2-arachidonoyl-glycerol (2AG), is currently thought to be the primary endogenous agonist for CB₁ and CB₂ receptors. The in vivo synthesis of 2AG involves, among other pathways, the action of phospholipase C to produce diacylglycerol, followed by the action of diacylglycerol lipase.

Monoglycerol lipase (“MGL”; a.k.a. monoacylglycerol lipase or “MAGL”) is a serine hydrolase that converts monoglycerides to fatty acid and glycerol. MGL is the primary enzyme for the degradation of 2-arachidonylglycerol (2-AG) in vivo (Dinh et al., PNAS USA 2002, 99(16): 10819-24; Quistad et al., Toxicol Appl Pharmacol. 2006, 211(1): 78-83, which is incorporated herein by reference). As discussed above, 2-AG is identified as one of the major endocannabinoids identified to date (Sugiura et al., Biochem Biophys Res Commun. 1995, 215(1): 89-97; Mechoulam et al., Biochem Pharmacol. 1995, 50(1): 83-90, which are incorporated herein by reference) and is a full agonist at both the CB₁ and CB₂ cannabinoid receptors. Furthermore, 2-AG exhibits several cannabinomimetic effects, including antinociception (Sugiura et al., J Biol. Chem. 1999, 274(5): 2794-801; Sugiura et al., J Biol. Chem. 2000, 275(1): 605-12; Sugiura and Waku, J Biochem (Tokyo) 2002, 132(1):7-12; Guindon et al., Br J. Pharmacol. 2007, 150(6): 693-701 2007, which are incorporated herein by reference). Micro-injection of a selective MGL inhibitor URB-602 into the periaqueductal grey region of rat brain enhanced stress-induced antinociception and this activity can be blocked by the CB₁ antagonist SR141716, suggesting a CB₁ receptor mediated process (Hohmann et al., Nature 2005, 435(7045):1108-1112; Connell et al., Neurosci Lett. 2006, 397(3): 180-4, which are incorporated herein by reference). Accordingly, inhibition of MGL activity, which concomitantly increases 2-AG levels, is implicated in the treatment of pain disorders (Hohmann, Br J Pharmacol. 2007, 150(6): 673-5, which is incorporated herein by reference; Guindon, 2007). Thus, MGL is an attractive drug target and specific inhibitors of MGL activity have immense therapeutic potential.

Conventional assays to monitor MGL activity are tedious, require the use of radioactive substrates (Dinh 2002, Ghafouri 2004, Brengdahl 2006), and are not particularly amenable to high-throughput screening (HTS). To date there is no real-time fluorescent assay reported for monitoring MGL activity. Due to the limitations of methods using radioactive agents for high-throughput screening (HTS), an assay for large libraries of chemical compounds that is simple, specific, and compatible with HTS is needed.

All references cited in this section above and throughout the entire disclosure below are incorporated herein by reference. However, Applicant reserves the right to challenge the veracity of any statement or statements made therein.

### SUMMARY

Disclosed is a novel fluorescence assay, which is useful to continuously monitor monoglycerol lipase (“MGL”) activity. The assay is simple in design and execution, and is amenable to HTS. The novel fluorescence assay is applicable to the identification of agents (e.g., compounds, therapeutic drugs, research reagents), which are capable of affecting the activity of MGL. Since MGL is an important enzyme related to endocannabinoid metabolism, the inventors envision that the assay is therefore applicable to the identification of agents that affect endocannabinoid activity. Agents that affect endocannabinoid activity are potential therapeutics.

In one aspect, the invention provides a method for detecting MGL activity, which comprises combining MGL with a fluorogenic substrate and detecting the resultant fluorescence output. The fluorescence signal is correlated to MGL activity. The fluorogenic substrates can be hydrolyzed by MGL to release a fluorescent molecule. In one embodiment, the fluorogenic substrate is 7-hydroxycoumarinyl-arachidonate. The assay method can be performed in wells of a multi-well plate, such as a 384-well plate, to accommodate high through-put screening.

In another aspect, the invention provides a method for identifying an agent or agents that modulate(s) MGL activity, which comprises combining an agent suspected of having an effect on MGL activity with MGL and a fluorogenic substrate; detecting the fluorescence output; determining MGL activity and comparing that activity to a baseline standard MGL activity. The baseline standard can be determined by combining the MGL and the fluorogenic substrate, but without the addition of the agent or agents; detecting the fluorescence output; and determining the baseline standard MGL activity. In one embodiment, the fluorogenic substrate is 7-hydroxycoumarinyl-arachidonate (“7-HCA”). The assay method can be performed in wells of a multi-well plate, such as a 384-well plate, to accommodate high through-put screening. In some embodiments, the method is deployed to identify agents that inhibit MGL activity by at least 10%, in other embodiments by at least 20%, and in yet other embodiments by at least 30% relative to the standard baseline MGL activity. The identified agent may have pro-endocannabinoid activity when administered to a patient in a therapeutically effective amount. An example of pro-endocannabinoid activity includes, but is not limited to, antinociception.

In another aspect, the invention provides novel polynucleotides, which are codon-optimized for expression in an ectopic protein expression system, and which encode the MGL polypeptides having or consisting of a sequence that is at least 37% homologous to SEQ ID NO:99. In one embodiment, the polynucleotide comprises a sequence of SEQ ID NO:1. In some embodiments, the polynucleotides are provided in cis (i.e., in the same polynucleotide strand) with a promoter, such as e.g., T7 promoter or cold-shock promoter A [cspA]). In one embodiment, the polynucleotides are inserted into a pCOLD™ II vector (Takara, Madison Wis.).

In another aspect, the invention provides kits useful in the determination of MGL activity. The kits contain an MGL with packaging materials and instructions.

In another aspect, the invention provides a method for making recombinant MGL in E. coli. In some embodiments, the method comprises inserting a polynucleotide, comprises a sequence that encodes a polypeptide having a sequence that is at least 37% homologous to SEQ ID NO:99 and which is codon-optimized for expression in E. coli, into a bacterial expression vector; and inducing the expression of the polypeptide. In one embodiment, the polynucleotide has a sequence of SEQ ID NO:1.

### BRIEF DESCRIPTION OF THE DRAWINGS

FIG. 1 depicts a reaction mechanism of 7-HCA hydrolysis by MGL. MGL catalyzes the hydrolysis of 7-HCA into arachidonic acid and 7-hydroxycoumarin (7-HC). Release of 7-HC, which is fluorescent, is monitored over time with excitation at about 355 nm and emission at about 460 nm.

FIG. 2 depicts the comparison of the hydrolysis of four potential fluorogenic substrates by MGL protein (7-HCA=7-Hydroxycoumarinyl-arachidonate, 7-HCL=7-Hydroxycoumarinyl-γ-linolenate, 7-HCH=7-Hydroxycoumarinyl-6-heptenoate and 7-AMCA=Arachidonyl-7-amino-4-methylcoumarin amide.)

FIG. 3, panel A depicts a SDS-PAGE gel of 10 μg of Ni⁺²-NTA column purified recombinant MGL-His6 protein which was separated on a 4-12% SDS-polyacrylamide gel electrophoresis and stained with Coomassie Blue. FIG. 3, panel B depicts a Western blot analysis of MGL-His6 protein which was transferred to a PVDF membrane and detected using Anti-His antibody. The arrows indicate the position of the MGL protein. Molecular mass standards are shown in the left margin.

FIG. 4 depicts a time course of 7-HCA hydrolysis by MGL protein.

FIG. 5 depicts the effect of concentration of MGL proteins on 7-HCA hydrolysis.

FIG. 6 depicts the effect of substrate concentration on MGL enzyme activity.

FIG. 7 depicts the effects of URB602, N-arachidonyl maleimide (NAM) and URB754 on MGL activity in the fluorescent assay.

FIG. 8 depicts MGL enzyme activity in varying assay conditions; panel A depicts pH dependence of the MGL activity; panel B depicts the effect of DMSO on hydrolysis of 7-HCA by MGL; and panel C depicts the effect of BSA on hydrolysis of 7-HCA by MGL FIG. 9 depicts an agarose gel electrophoresis analysis of PCR-directed C-HMGL cDNA synthesis.

FIG. 10 depicts the expression of codon-optimized and non-optimized HMGL cDNA in E. coli BL21 cells.

FIG. 11 depicts the effect of IPTG concentration on C-HMGL synthesis in E. coli BL21 cells.

FIG. 12 depicts the effect of IPTG concentration on N-HMGL synthesis in E. coli BL21 cells.

FIG. 13 depicts the effect of IPTG concentration on C-HMGL synthesis in E. coli BL21 cells.

FIG. 14 depicts the effect of IPTG concentration on N-HMGL synthesis in E. coli BL21 cells.

FIG. 15 depicts the time course of C-HMGL expression in E. coli BL21 cells using pCOLD™ II vector.

FIG. 16 depicts the time course of C-HMGL expression in E. coli BL21 cells using pET16b(+) vector.

FIG. 17 depicts a SDS-PAGE analysis of Ni-IDA column purified HMGL protein samples.

FIG. 18 depicts a functional activity of HMGL protein.

### DETAILED DESCRIPTION OF THE INVENTION

### Fluorescence Method for Determining MGL Activity

In one embodiment, the invention is directed to a fluorescence-based method for determining the activity of a monoglycerol lipase (a.k.a. monoacylglycerol lipase). A monoglycerol lipase (“MGL”) can be combined with a fluorogenic substrate in a suitable buffer. The resultant mixture is subjected to light of a wavelength sufficient to excite the hydrolyzed fluorescent product, and the fluorescence emission is detected. MGL activity is determined by converting the detected fluorescence into activity units.

This fluorescence-based method lends itself well to high through-put screening (“HTS”) applications as well as traditional bench research, replacing the unwieldy radioactive tritiated glycerol release assays. The method can be performed in any system, such as for example in a test tube, in a well or wells of a multi-well plate (e.g., a 384-well microtitre plate), and/or in a hanging drop. This embodiment is applicable to the identification of agents that modulate MGL activity. Agents identified using this method can have a potential therapeutic effect on endocannabinoid function in a patient. As used herein, “agents” includes compounds, drugs, pro-drugs, drug-candidates, libraries of compounds, subsets of libraries of compounds, biomolecules, proteins, peptides, nucleic acids, salts, lipids and the like.

In some embodiments, the monoglycerol lipase is a human MGL, although any protein from any source having EC 3.1.1.23 activity is applicable to this invention. In one embodiment, MGL has a sequence that is at least about 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% homologous to SEQ ID NO:99. In some embodiments, the MGL is included in the reaction mixture at a concentration of from about 0.1 ng to about 100 ng of MGL per 50 μl of total volume of mixture, in other embodiments from about 1 ng to about 10 ng of MGL per 50 μl of total volume of mixture, and in yet other embodiments at about 3 ng of MGL per 50 μl of total volume of mixture.

The fluorogenic substrate can have a fatty acyl part joined to a fluorescent moiety via an ester linkage. In this way, the fluorogenic substrate mimics a natural lipid substrate for MGL. The fluorogenic substrate does not have the same fluorescence spectrum as the fluorescent moiety, which allows for the fluorescence detection of the fluorescent moiety. The fluorescent moiety is detected upon its release due to the carboxylic ester hydrolase activity of MGL. The amount of fluorescence moiety detected can thus be correlated to MGL activity.

Fluorogenic moieties can be any fluorescent molecule that can be joined to a hydrocarbon chain via a carboxylic ester linkage, such as coumarin and its analogs (e.g., 7-hydroxycoumarin), pyridine, and pyridine analogs (e.g., 3-amino-4-methylpyridine, 3-amino-6-methylpyridine, 3-amino-2-fluoropyridine, 5-amino-2-fluoropyridine, 5-amino-2-methoxypyridine, 3-amino-2-methoxypyridine, 3-amino-6-methoxy-2-picoline, and 3-aminopyridine). In some embodiments, the fluorescent moiety is 7-hydroxycoumarin.

The fatty acyl part of a suitable fluorogenic substrate can be a long chain fatty acyl group, such as for example an arachidonate, γ-linolenate, or 6-heptenoate. However, the skilled artisan would readily recognize that other fatty acyl groups (fatty acids) can be used while preserving the ability of the fluorogenic substrate to serve as substrate for an MGL. In some embodiments, the fatty acyl part is arachidonate. In one embodiment, the fluorogenic substrate is 7-hydroxycoumarinyl-arachidonate. The fluorogenic substrate can be included in a reaction mixture at a concentration of from about 0.1 μM to about 100 μM, or from about 1 μM to about 20 μM, or at about 10 μM. The skilled artisan would readily recognize that the fluorogenic substrate should not be limiting to the progression of the carboxylic ester hydrolase reaction.

The skilled artisan would readily know what buffer system or systems would allow activity of a lipase, while accommodating the substrate and products. In one aspect, a suitable buffer can have a pH of between about pH 4 and 12, or between about pH 8 and 10, or about pH 8. A suitable pH can be obtained by using 4-(2-hydroxyethyl)-1-piperazineethanesulfonic acid (“HEPES”), at a concentration of 50 mM. However, the buffer system is not limited to this embodiment. The buffer may optionally contain dimethyl sulfoxide (“DMSO”) and/or bovine serum albumin (“BSA”). DMSO may be present at a concentration of from about 0.5% to about 10%, or about 10%. BSA may be present at a concentration of from about 0.001% to about 10%, or about 0.1%.

In one aspect, the MGL and fluorogenic substrate are combined in a buffer to form a mixture, and the mixture is allowed to incubate for a time and at a temperature sufficient to allow for the hydrolysis of the fluorogenic substrate and subsequent release of fluorescent moiety product. In some embodiments, the time is from about 30 minutes to about 120 minutes, from about 30 minutes to about 90 minutes, or about 60 minutes. In some embodiments, the temperature is from about 15° C. to about 40° C., from about 20° C. to about 30° C., or about 25° C. In one embodiment, the mixture is allowed to incubate for about 60 minutes at about 25° C.

The resultant mixture is subjected to light of an appropriate wavelength to excite the hydrolyzed fluorescent product, and the fluorescence emission is detected. The skilled artisan in the practice of the invention determines the appropriate excitation and emission wavelengths by ascertaining the fluorescence spectra of the fluorescent moiety employed in the method. For example, when 5-amino-2-methoxypyridine is used, the excitation light may include light having a wavelength of about 250 nm to about 350 nm, optimally of about 302 nm, with the fluorescence emission detected at 360 nm to about 460 nm, optimally at about 396 nm. In the case in which 7-hydroxycoumarin (a.k.a. umbelliferone) is used, the excitation light may include light having a wavelength of about 290 nm to about 370 nm, optimally of about 325 nm or about 355 nm, with the fluorescence emission detected at 390 nm to about 560 nm, optimally at about 452 nm or about 460 nm. In one aspect, wherein 7-hydroxycoumarin is used as the fluorescent moiety, the mixture is excited at about 355 nm and the fluorescence is detected at about 460 nm.

In some embodiments, MGL activity is expressed as A=x/t/y; wherein A is activity, x is product concentration, t is time, and y is amount of enzyme. In one embodiment, activity is expressed as picomoles of fluorescent moiety released via hydrolysis of the fluorogenic substrate per minute per milligram of MGL.

### Identifying Agents that Modulate MGL

In another aspect, the invention is provides a method for identifying agent(s) that are capable of modulating MGL activity. Since MGL has been identified as important to the metabolism of the endocannabinoid 2-arachidonylglercerol (“2-AG”), those agents identified have potential as therapeutic agents that affect endocannabinoid activity. Endocannabinoid activity is known to affect addiction behavior, eating behavior, nociception, memory, attenuation of multiple sclerosis, oligodendrocyte function, neurogenesis and control of pregnancy. Thus, agent(s) that inhibit MGL activity are expected to have potential as therapeutic drugs, such as for example antinociceptive agents. As used herein, “modulation” means that an agent can either increase the activity of an MGL, decrease the activity of MGL, inhibit the activity of MGL or block the activity of MGL. Agent is meant in a broad sense to include any and all materials, which may be tested for MGL modulation activity, and includes, but is not limited to, compounds, small molecules, biological molecules, salts, metals, combinations of compounds, and libraries of compounds. The term small molecule is used to refer to any and all molecules that have a relatively low molecular weight. Typically, small molecules are monomeric and have a molecular weight of less than about 1500 g/mol. In some embodiments, the agent is capable of inhibiting the hydrolysis of 2-arachidonylglercerol (“2-AG”) in vivo. In one embodiment, the agent is capable of increasing the level of an endocannabinoid in a physiological system.

The method comprises the steps of combining an MGL, a fluorogenic substrate, and an agent, to form a mixture. The mixture is then subjected to light that is capable of exciting a fluorescent moiety that can be released from the fluorogenic substrate via MGL mediated hydrolysis. Fluorescent light is detected and correlated to MGL activity. The modulating effect that the agent has on MGL activity is quantified by comparing the activity of MGL in the absence of an agent, to the activity of the MGL in the presence of the agent. Activity is generally expressed as A=x/t/y; wherein A is activity, x is product concentration, t is time, and y is amount of enzyme. Activity can also be expressed as picomoles of fluorescent moiety released via hydrolysis from the fluorogenic substrate per minute per milligram of MGL. In one aspect, those agents that modulate MGL activity by a difference of 20% or more are identified. In another aspect, those agents that modulate MGL activity by a difference of 30% or more are identified. The method can be applied to a high throughput screen (“HTS”) to identify agents that potentially can modulate MGL activity. In the HTS aspect, reactions can be performed in individual wells of a microtitre plate, such as a 384-well plate.

While any enzyme having EC 3.1.1.23 activity can be used in the practice of this method, in some embodiments, the MGL is a recombinant human MGL, which can be produced in any one or more of protein expression systems by inserting a polynucleotide encoding the recombinant MGL polypeptide into an expression vector, which is then put into a host cell. One suitable expression system is a bacterial expression system described in more detail below. In some embodiments, the expression system is an E. coli expression system. The expression system can comprise a cold-shock induced E. coli system using a pCOLD™ vector and an E. coli BL21 host strain. A cold-induced system can improve protein folding and allow for improved solubility and stability of the recombinant protein. In one embodiment, the polynucleotide encoding the recombinant MGL is codon-optimized for expression in E. coli, such as e.g. SEQ ID NO:1.

The fluorogenic substrate used in this method is capable of being hydrolyzed by an enzyme having carboxylic ester hydrolase activity, such that one of the products is a fluorescent moiety that can be detected via fluorescence. The fluorogenic substrate can comprise two parts, a fluorescent moiety and a fatty acyl group, joined together via a carboxylic ester bond. When the bond is hydrolyzed, a free fatty acid and a fluorescent compound are released. In some embodiments, the fatty acyl group is a long chain fatty acyl, such as arachidonate, γ-linolenate, and 6-heptenoate. In one embodiment, the fatty acyl group is arachidonate. In some embodiments, the fluorescent moiety is any one of coumarin, pyridine, and their respective analogs. In one embodiment, the fluorescent moiety is 7-hydroxycoumarin (a.k.a. umbelliferone). Taken together, in some embodiments, the fluorogenic substrate can be any one of 7-hydroxycoumarinyl-arachidonate, 7-hydroxycoumarinyl-γ-linolenate, and 7-hydroxy-coumarinyl-6-heptenoate. In one embodiment, the fluorogenic substrate is 7-hydroxycoumarinyl-arachidonate. Hydrolysis of 7-hydroxycoumarinyl-arachidonate by MGL yields the fluorescent molecule 7-hydroxycoumarin, which has an absorbance maximum at about 325 nm and a fluorescence emission maximum at about 452 nm.

In the case wherein 7-hydroxycoumarin (a.k.a. umbelliferone) is used as the fluorescent moiety, the excitation light may include light having a wavelength of about 290 nm to about 370 nm, optimally of about 325 nm or about 355 nm, with the fluorescence emission detected at 390 nm to about 560 nm, optimally at about 452 nm or about 460 nm. In one aspect of this case, wherein 7-hydroxycoumarin is used as the fluorescent moiety, the mixture is excited at about 355 nm and the fluorescence is detected at about 460 nm.

In one aspect of the invention, the agent, MGL and fluorogenic substrate are combined in a buffer to form a reaction mixture. The term “reaction mixture” may be used interchangeably with mixture, resultant mixture, screening mixture and/or assay mixture. In some embodiments, the buffer has a pH and ionic strength that allows for at least 10% MGL activity; for example from about pH 8 to about pH 10, or about pH 8. A buffer can contain an organic buffer, such as 4-(2-hydroxyethyl)-1-piperazineethanesulfonic acid (“HEPES”). In one embodiment, the buffer comprises HEPES at a concentration of about 50 mM.

The fluorogenic substrate can be included in the reaction mixture from about 1 μM to about 20 μM, or at about 10 μM. The MGL enzyme can be included in the reaction mixture of some embodiments from about 0.1 ng to about 100 ng of MGL per 50 μl of total volume of mixture, from about 1 ng to about 10 ng of MGL per 50 μl of total volume of mixture, or at about 3 ng of MGL per 50 μl of total volume of mixture.

In another aspect, the buffer can contain additional ingredients. One such additional ingredient is dimethyl sulfoxide (“DMSO”), which can be included at a concentration of from about 0.5% to about 10%, or at about 10%. Another such additional ingredient is bovine serum albumin (“BSA”), which can be at a concentration of from about 0.001% to about 1%, or at about 0.1%. Other suitable additional ingredients can be readily identified by one of ordinary skill in the art.

In another aspect, the reaction mixture is incubated for a length of time and temperature to allow for sufficient MGL activity to have occurred to render reliable results. In some embodiments, the incubation time is from about 30 minutes to about two (2) hours, or about 60 minutes. In some embodiments, the incubation temperature is from about 20° C. to about 30° C., or about 25° C.

The various reaction, incubation and buffer conditions can be changed to suit the practitioner in the practice of this invention. However, it is important to note that the reaction parameters used in both the presence and absence of the agent should be very similar to identical or allow for meaningful comparison.

### Monoglycerol Lipase Polypeptides

In another embodiment, the invention is directed to a recombinant human MGL, which is produced by any suitable protein expression system. Non-limiting examples of suitable protein expression systems include the Pichia yeast system, bacterial systems, insect bacculovirus systems, Leischmania systems, mammalian systems such as the CHO cell system, and transgenic plant and animal systems.

In some embodiments, the recombinant human MGL is produced in a bacterial expression system. The recombinant human MGL is produced by inserting a polynucleotide, which encodes the recombinant human MGL, into a bacterial expression vector, placing the resultant recombinant expression-vector-with-insert into a bacterial host, which then produces the recombinant human MGL. Bacterial expression vectors and bacterial hosts are well-known in the art. Non-limiting examples of bacterial hosts include Bacillus brevis, Bacillus megaterium, Bacillus subtilis, Caulobacter crescentus, and various strains of Escherichia coli, such as BL21 and K12 and their respective derivatives (e.g., Origami, Rosetta, AD494). Non-limiting examples of bacterial expression vectors include those that utilize promoters such as lac, tac, trc, T7, phage promoter p_{L}, tetA, araBAD, rhaP_{BAD}, and cspA. For an overview of bacterial expression hosts and vectors, see Terpe, K, “Overview of bacterial expression systems for heterologous protein production: from molecular and biochemical fundamentals to commercial systems,” Appl. Microbiol. Biotechnol (2006) 72:211-222, and references therein, which are incorporated herein by reference.

In some embodiments, the recombinant MGL is produced in an E. coli expression system. Those E. coli host strains include for example K-12, AD494, BL21, BL21 trxB, BL-21 CodonPlus™-RIL, BL-21 CodonPlus™-RP, BLR, B834, C41, C43, HMS 174, JM 83, ORIGAMI™, ORIGAMI™ B, ROSETTA™, and ROSETTA™-gami. In one embodiment, the E. coli host strain is BL21. While it is envisioned that any E. coli expression system can be used in the practice of this invention, in one aspect, the E. coli expression system is a cold expression system, which allows for improved solubility and stability of recombinant protein. Such a system utilizes a cold shock promoter, e.g., cspA, to drive expression of the recombinant MGL polypeptide. In one embodiment of the cold driven expression system, the expression vector is a pCOLD™ II vector (Takara, Madison Wis.), which is described in detail in Qing et al., “Cold-shock induced high-yield protein production in Escherichia coli,” Nature Biotechnology 22, 877-882, 2004, which is incorporated herein by reference. The pCOLD™ II vector has a sequence described in GENBANK® as GenBank Accession No. AB186389. The E. coli host can enable expression of heterologous proteins.

An exemplary amino acid sequence of a human MGL is provided in SEQ ID NO:99. The MGL need not be restricted human MGL, and can be any protein having MGL activity, with sequences as diverse as 37% homology or more with respect to SEQ ID NO:99. Thus, in some embodiments, the recombinant MGL polypeptide has a sequence that is at least about 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% homologous to SEQ ID NO:99. Table 1 depicts examples of MGL sequences that are suitable for use in this invention.

TABLE 1
MGL EXAMPLES

| Genus | GENBANK ID¹ | Homology to Human MGL |
|---|---|---|
| Homo | NP 001003794.1 | 100% |
| Canis | XP 533717.2 | 92% |
| Equus | XP 001488869.1 | 90% |
| Macaca | XP 001099305.1 | 88% |
| Ornithorynchus | XP 001506833.1 | 86% |
| Bos | XP 581556.3 | 85% |
| Mus | NP 035974.1 | 84% |
| Rattus | NP 612511.1 | 83% |
| Gallus | XP 414365.2 | 70% |
| Xenopus | NP 001087903.1 | 60% |
| Danio | NP 956591.1 | 56% |
| Monkeypox | NP 536458.1 | 47% |
| Tanapox | YP 001497005.1 | 37% |

¹The GenBank sequences are incorporated herein by reference.

Percent homology with respect to the SEQ ID NO:99, or other reference MGL sequences (for example, those in Table 1) is defined as the percentage of amino acid residues in a candidate sequence that are identical with the amino acid residues in the SEQ ID NO:99 or other reference MGL sequence, respectively, after aligning the sequences and introducing gaps, if necessary, to achieve the maximum percent sequence identity, and not considering any conservative substitutions as part of the sequence identity. Alignment for purposes of determining percent homology can be achieved in various ways that are within the skill in the art, for instance, using publicly available computer software such as BLAST, BLAST-2, ALIGN or Megalign (DNASTAR) software.

The recombinant MGL can be used in any assay, such as the fluorescence MGL assay and/or the screen for agents that modulate MGL activity, both of which are disclosed herein (supra). In some embodiments of said assay or screen, the MGL can be included in the mixture at a concentration of from about from about 1 ng to about 10 ng of MGL per 50 μl of total volume of mixture, or about 3 ng of MGL per 50 μl of total volume of mixture.

### Polynucleotides Encoding Monoglycerol Lipase Polypeptides

In another aspect, the invention is directed to polynucleotides that encode a recombinant monoglycerol lipase, wherein the polynucleotides comprise codons that are optimized for expression of the polynucleotide in a biological host system. Modifying codons in a given polynucleotide to include those favored by a particular host is called codon optimization. Several programs are available for this process, including web-based packages, in which a polynucleotide sequence is input, a codon usage table is selected and input for the heterologous species and/or strain to be used for polypeptide expression, and the output is a codon-optimized polynucleotide. Exemplar programs are described in Gao et al., “UpGene: Application of a web-based DNA codon optimization algorithm,” Biotechnol. Prog. 2004, March-April; 20(2):443-8; Barrett et al., “Optimization of Codon Usage of Poxvirus Genes allows for Improved Transient Expression in Mammalian Cells,” Virus Genes. August 2006; 33(1): 15-26; Kim et al., “Codon optimization for high-level expression of human erythropoietin (EPO) in mammalian cells,” Gene. 1997; 199: 293-301; Ravi et al., “Codon Optimization for DNA Vaccines and Gene Therapy Using Pattern Matching,” CSB. August 2003; Jorgensen et al., “Heterogeneity in Regional GC Content and Differential Usage of Codons and Amino Acids in GC-Poor and GC-Rich Regions of the Genome of Apis mellifera,” Molecular Biology and Evolution. December 2006.

TABLE 2
CODON USAGE FOR E. COLI STRAIN K12

| | | | | | | | | | | | |
|---|---|---|---|---|---|---|---|---|---|---|---|
| UUA | L | 0.13 | CUU | L | 0.10 | AUU | I | 0.51 | GUU | V | 0.26 |
| UUG | L | 0.13 | CUC | L | 0.10 | AUC | I | 0.42 | GCU | A | 0.16 |
| UUU | F | 0.57 | CUA | L | 0.04 | AUA | I | 0.07 | GAU | D | 0.63 |
| UUC | F | 0.43 | CUG | L | 0.50 | ACU | T | 0.17 | GGU | G | 0.34 |
| UCU | S | 0.15 | CCU | P | 0.16 | ACC | T | 0.44 | GUC | V | 0.22 |
| UCC | S | 0.15 | CCC | P | 0.12 | ACA | T | 0.13 | GCC | A | 0.27 |
| UCA | S | 0.12 | CCA | P | 0.19 | ACG | T | 0.27 | GAC | D | 0.37 |
| UCG | S | 0.15 | CCG | P | 0.53 | AAU | N | 0.45 | GGC | G | 0.40 |
| UAU | Y | 0.57 | CAU | H | 0.57 | AAC | N | 0.55 | GUA | V | 0.15 |
| UAC | Y | 0.43 | CAC | H | 0.43 | AGU | S | 0.15 | GCA | A | 0.21 |
| UGU | C | 0.44 | CGU | R | 0.38 | AGC | S | 0.28 | GAA | E | 0.69 |
| UGC | C | 0.56 | CGC | R | 0.40 | AAA | K | 0.77 | GGA | G | 0.11 |
| UAA | * | 0.63 | CGA | R | 0.06 | AAG | K | 0.23 | GUG | V | 0.37 |
| UGA | * | 0.29 | CGG | R | 0.10 | AGA | R | 0.04 | GCG | A | 0.36 |
| UAG | * | 0.07 | CAA | Q | 0.35 | AGG | R | 0.02 | GAG | E | 0.31 |
| UGG | W | 1.00 | CAG | Q | 0.65 | AUG | M | 1.00 | GGG | G | 0.15 |

Column 1 = codon triplet, column 2 = amino acid, column 3 = frequency of use per amino acid.

As mentioned in the preceding section, in some embodiments, the system for producing the recombinant MGL is an E. coli system, and therefore in some aspects the polynucleotide is optimized for translation in an E. coli bacterium. An example of such a codon-optimized polynucleotide is depicted in SEQ ID NO:1 (FIG. 9). By way of example, Table 2 presents codon usage data for the common bacterial expression vector E. coli strain K12.

In one embodiment of the invention, the polynucleotide comprises a promoter for driving expression of the recombinant MGL in the host expression system. In some embodiments, the promoters are functional in an E. coli expression system, examples of which include lac, tac, trc, T7, phage promoter p_{L}, tetA, araBAD, rhaP_{BAD}, and cspA (Terpe 2006). In some embodiments, the promoter is a cold shock promoter cspA, which has a 5′ UTR located upstream (i.e., 5-prime) of the MGL coding sequence and a 3′ UTR located downstream (i.e., 3-prime) of the MGL coding sequence, which comprises a transcription terminator site. One embodiment comprises is a combination promoter containing both a lac promoter, which is controllable through IPTG, and the cspA promoter. In one aspect, the lac promoter is located upstream of the cspA 5′ UTR.

In another embodiment of the invention, the polynucleotide can comprise element(s) to increase the production of recombinant MGL. Examples of applicable elements include a conversion of the AAGG to GAGG of the Shine-Delgarno sequence, inclusion of the translational enhancer (SEQ ID NO:100 and SEQ ID NO:101) element as described in Etchegaray and Inouye, “Translational Enhancement by an Element Downstream of the Initiation Codon in Escherichia coli,” J Biol Chem, Vol. 274, Issue 15, 10079-10085, Apr. 9, 1999; and the downstream box (“DB”) of Sprengart et al., “The downstream box: an efficient and independent translation initiation signal in Escherichia coli,” EMBO J. 1996 Feb. 1; 15(3): 665-674, which are incorporated herein by reference. Translation enhancer elements (“TEE”) are also described in Makrides, S. C., “Strategies for Achieving High-Level Expression of Genes in Escherichia coli,” Microbiological Reviews, September 1996, pp. 512-539; and Mauro et al., WIPO publication number WO/2007/025008, which are incorporated herein by reference. In one embodiment, the polynucleotide comprises the translation enhancer (SEQ ID NO:100) element, which is located between the cspA 5′ UTR and the initiator codon of the sequence encoding the recombinant MGL polypeptide.

In yet another embodiment, the polynucleotide contains a nucleotide sequence that encodes a molecular tag useful in the tracking and/or purification of the resultant recombinant MGL polypeptide. The nucleotide sequence that encodes a molecular tag is in frame with the coding sequence for the MGL polypeptide, and may be upstream (i.e., to be located at the N-terminus of the recombinant MGL polypeptide) or downstream (i.e., to be located at the C-terminus of the recombinant MGL polypeptide) of the MGL coding sequence. Non-limiting examples of molecular tags include biotin carboxyl carrier protein (“BCCP”), c-myc, calmodulin, FLAG, hexahistidine (“His₆”), decahistidine (“His₁₀”), glutathione-s-transferase (“GST”), green fluorescence protein (“GFP”), thioredoxin and streptavidin. In one embodiment, the molecular tag is a His₆-tag.

In yet another embodiment, the polynucleotide is an expression vector containing an MGL-encoding polynucleotide inserted into its multiple cloning site. In one embodiment, the MGL-encoding polynucleotide is codon-optimized for expression in E. coli, an example of which is provided as SEQ ID NO:1. The mammalian version of the sequence is depicted in SEQ ID NO:2, which may also be used in the practice of this invention. Exemplary expression vectors include, but are not limited to pQE80L-D33, pQE80L, pGST-1, pGEX-6P-1, pHis-2, pBAT4, pGST-2, pNusA, pASK75, and pMBP, which are described in Korf et al., “Large Scale Protein Expression for Proteomic Research,” Proteomics 2005, 5:3571-3580, which is incorporated herein by reference; pDEST17, pTH19, pTH26, pTH24, pET-15b, and pET-DEST42, which are described in Woestenenk et al., “His tag effect on solubility of human proteins produced in Escherichia coli: a comparison between four expression vectors,” J. Structural & Functional Genomics 5:217-229, 2004, which is incorporated herein by reference; and pCOLD™ I, pCOLD™ II, pCOLD™ III, and pCOLD™ IV, which are described in Qing et al. 2004, which is incorporated herein by reference. In some embodiments, the expression vector can be any pCOLD™ vector, or a pCOLD™ II vector. In one embodiment, the polynucleotide is a pCOLD™ II vector containing SEQ ID NO:2 inserted in-frame into the multiple cloning site (“MCS”).

### Process for Manufacturing a Recombinant MGL

In another aspect, the invention provides methods of making functional recombinant MGL protein. A codon-optimized polynucleotide encoding an MGL polypeptide is inserted into an expression vector, which is in turn inserted into a host cell that expresses the recombinant MGL polypeptide. The recombinant MGL polypeptide can be purified using various methods known in the art, such as for example ammonium sulfate precipitation, affinity matrix binding, molecular sieve chromatography, ion exchange chromatography, HPLC, FPLC and combinations thereof. In some embodiments, the polynucleotide is codon-optimized for expression in E. coli, and the host cell is an E. coli strain BL21 (D3). In one embodiment, the polynucleotide has a sequence described in SEQ ID NO:1.

In one embodiment, the process comprises the steps (a) inserting a polynucleotide, which encodes an MGL polypeptide and has a sequence comprising codons optimized for expression in an E. coli bacterial host system, into a bacterial expression vector; (b) putting the bacterial expression vector into E. coli; and (d) inducing expression of the MGL polypeptide under cold conditions (e.g., 16° C.). Makrides (1996), which is incorporated herein by reference, gives general guidance to high-level expression of heterologous proteins in E. coli.

### Recombinant MGL Kits

In yet another embodiment, the invention is directed to kits useful for measuring MGL activity or for screening for an agent or agents that modulate MGL activity. The kits can contain an MGL, instructions and packaging materials. The instructions can instruct the end-user on performing a fluorescence assay to detect MGL activity. Other materials that may be included in the kits are, for example, buffers, such as HEPES, assay components, such as a fluorogenic substrate like 7-hydroxycoumarinyl-arachidonate, additional proteins, such as albumin, and/or solubilization agents such as DMSO.

### EXAMPLES

The following examples are included to demonstrate particular embodiments of the invention. It should be appreciated by those of skill in the art that the techniques disclosed in the examples represent techniques discovered by the inventor to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

### Example 1

### Fluorescence Assay

Purified recombinant human MGL protein and 7-Hydroxycoumarinyl-arachidonate (7-HCA), a fluorogenic substrate for MGL, were employed in the assay. MGL protein catalyzes the hydrolysis of 7-HCA to generate arachidonic acid and the highly fluorescent 7-hydroxycoumarin (7-HC). Release of HC was monitored continuously using a fluorometer. MGL protein catalyzed the hydrolysis of 7-HCA with an apparent K_{M} of 9.8 μM and Vₘₐₓ of 1.7 μmoles min⁻¹ mg protein⁻¹. The successful cleavage of 7-HCA depends upon active MGL, as assay buffer alone or heat-denatured MGL protein had no significant activity against 7-HCA. Furthermore, MGL activity was inhibited by the specific inhibitor URB-602 as well as by N-Arachidonyl maleimide (NAM) in a dose-dependent manner with IC₅₀ values of 3.1 μM and 155 nM respectively.

The assay was further optimized under different conditions, including pH range, BSA protein and DMSO concentrations. The assay was found to be reproducible, having Z-PRIME values ranging from 0.7-0.9, and is therefore suitable for HTS.

### Materials

7-Hydroxycoumarinyl-arachidonate, 7-Hydroxycoumarinyl-Y-linolenate, 7-Hydroxy-coumarinyl-6-heptenoate and Arachidonyl-7-amino-4-methylcoumarin amide were from Biomol Research Lab (Plymouth Meeting, Pa.). 2-Arachidonyl glycerol, [glycerol 1, 3-³H] (20-40 Ci/mmol) was purchased from American Radiolabled Chemicals, Inc (St. Louis, Mo.). URB602, URB754 and N-Arachidonyl Maleimide were purchased from Cayman Chemical Company (Ann Arbor, Mich.). Oligonucleotides for human MGL gene synthesis were purchased from IDT Technologies (Coralville, Iowa). PCR enzymes were purchased from Stratagene (La Jolla, Calif.) and Takara-Mirus (Madison, Wis.). Restriction enzymes were purchased from New England Biolabs (Ipswich, Mass.). IPTG was purchased from Invitrogen (Carlsbad, Calif.). Chemically competent host strains E. coli DH5a, BL21 and TOP10 were purchased from Invitrogen. The pCOLD™ II expression vector was purchased from Takara-Mirus and pET16b (+) expression vector was purchased from Novagen (Madison, Wis.).

### MGL Gene Synthesis and Expression Construct

The MGL gene was synthesized according to the following procedure. Briefly, a polymerase chain reaction (“PCR”) was carried out with a mixture of forward and reverse primers spanning the entire coding region (4 mM each) with a DNA polymerase for 30 cycles in the gene assembly step. SEQ ID NO:3-26 are codon-optimized human MGL (“C-HMGL”) forward sense primers, SEQ ID NO:51-74 are non-codon-optimized human MGL (“N-HMGL”) forward sense primers, SEQ ID NO:27-50 are C-HMGL reverse antisense primers, and SEQ ID NO:75-98 are N-HMGL reverse antisense primers. For gene amplification, a second PCR was carried out using the product from previous gene assembly reaction as a template and with the two outermost oligonucleotides at 1 mm concentration for 30 cycles. All PCR generated MGL cDNA fragments were agarose gel purified using a purification kit (Qiagen, Valencia, Calif.) and then ligated into the PCR-blunt vector (using the Zero Blunt PCR cloning kit, Invitrogen) and then used to transform TOP10™ chemically competent E. coli cells according to the manufacturer's protocol (Invitrogen). The resulting clones were selected on LB agar plates containing 50 mg/ml of kanamycin (Teknova, Hollister, Calif.) and constructs were verified by DNA sequencing.

To generate the plasmids for E. coli expression, the sequence verified MGL cDNA fragment was digested with NdeI and XhoI and then ligated into either the pCOLD™ II or pET16b (+) expression vectors (Novagen,) digested with NdeI/XhoI, and then used to transform E. coli BL21 cells. The resulting clones were selected on LB agar plates containing 50 mg/ml of carbenicillin. The recombinant plasmid was verified by restriction digestion analyses followed by DNA sequencing.

### Expression of HMGL in BL21 E. Coli Cells

Colonies were picked from LB agar plates containing 50 mg/ml carbenicillin plates (Teknova) and were grown in LB medium containing the same antibiotic concentration as in the plate. Cells were grown at 37° C. with aeration until OD₆₀₀=0.5-0.7 was reached. The cell cultures were equilibrated to 16° C. for 20-30 min (for expression using pCOLD™ II vector) and then MGL expression was induced by adding isopropyl β-D-galactoside (IPTG) to a final concentration of 1 mM for 3-72 hours. 1 liter of the induced cultures were pelleted by centrifugation (14,000×g for 5 min) and MGL-His6 was purified via Ni²⁺-nitrilotriacetate resin-based chromatography (TALON™ Metal Affinity Resins Protein purification kit from BD Biosciences, Palo Alto, Calif.). MGL-His6 proteins were analyzed by NuPAGE 4-20% Bis-Tris gradient SDS-PAGE (Invitrogen) and Western blot using a mouse-specific monoclonal antibody raised against the His tag (Invitrogen).

### 7-HC-Arachidonate Hydrolysis by MGL Protein

The assay was performed in a 384-well black plate (NUNC) in a total volume of 50 μl. A typical assay consisted of 3 ng of MGL protein, 10 mM 7-HCA substrate (prepared in DMSO) in assay buffer consisting of 50 mM HEPES, pH 8, 1 mM EDTA, and 5 ml of inhibitor solution or DMSO (10% final concentration). The components were mixed and incubated on a shaker for 10-60 min and the plate was read in a fluorescence plate reader (SPECTRAMAX™-Gemini; Ex: 355 nm; Em: 460 nm). The reaction was carried out at 25° C., and the fluorescence was monitored in the kinetic mode. The fluorescence units were converted to amount of product based on the standard curve generated with 7-HC. The data were analyzed using GRAPHPAD™ Prism Version 3.02. The observed rate constants (apparent KM) were calculated using GRAPHPAD™ Prism 3.02 based on a Michaelis-Menten (one site binding hyperbola) equation Y=BmaxX/[Kd+X].

For measuring MGL inhibition by compounds, 3 ng protein per assay were incubated with indicated concentrations of the compound in the presence of 10 mM 7-HCA for 60 minutes. The reactions were stopped and processed as described above. Results are presented as the mean±S.E. of triplicate measurements from at least two independent experiments. The IC50 values were calculated by GRAPHPAD™ Prism 3.02 program using a sigmoidal dose-response equation.

### [³H]-2-AG Hydrolysis by MGL Protein

3 ng of MGL protein were incubated with indicated concentrations of 2-AG, [glycerol-1-³H] for 30 min at 25° C. in buffer containing 50 mM Tris-HCl pH 8, 1 mM EDTA, 10% DMSO and compound. The reaction was terminated by the addition of 2 volumes of chloroform/methanol (1:1, v/v) after 60 minutes. The samples were vortexed and centrifuged at 1000 g for 3 minutes. The aqueous phase, containing [³H]-glycerol, was collected and measured by liquid scintillation counting. Results are presented as the mean±S.E. of triplicate measurements from at least two independent experiments.

### Western Blot

1 mg of MGL-His6 protein purified from E. coli was analyzed by SDS-PAGE on 4-12% polyacrylamide gels (Invitrogen) in duplicate. One of the gels was stained with Coomassie blue, while the other was blotted on to a PVDF membrane. MGL-His6 protein was detected by staining with the mouse anti-His monoclonal antibody (Invitrogen, 1:200 dilution) followed by incubation with an IRDye 800CW Goat anti mouse secondary antibodies in 1:10,000 dilution (LI-COR, Lincoln, Nebr.). The signal was detected by using ODYSSEY™ infrared Imaging System according to the manufacturer's protocol.

### Calculation of Z-PRIME Factor

For calculating the Z-PRIME value (23), which is a measure of the quality of the assay and suitability for HTS, the assay was performed as described above, using assay buffer without MGL protein as negative control, in multiple 384-well plates. The Z-PRIME factor was calculated according to Equation 1, where σ_{c+} and σ_{c-} are standard deviations of positive and negative controls, and μ_{c+} and μ_{c-} are mean of positive and negative controls respectively.

$${Z}^{’} = 1 - \frac{( 3 {σ}_{c +} + 3 {σ}_{c -} )}{\vert {μ}_{c +} - {μ}_{c -} \vert} \qquad Equation 1$$

### MGL Fluorogenic Substrate Selection and Assay Design

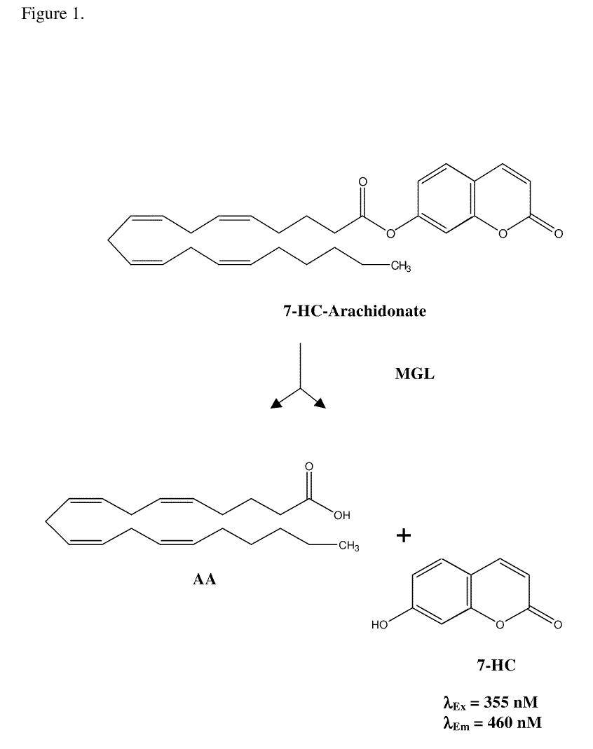

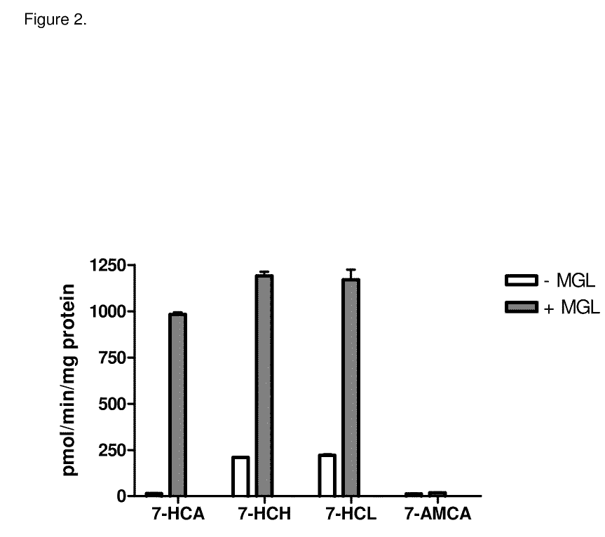

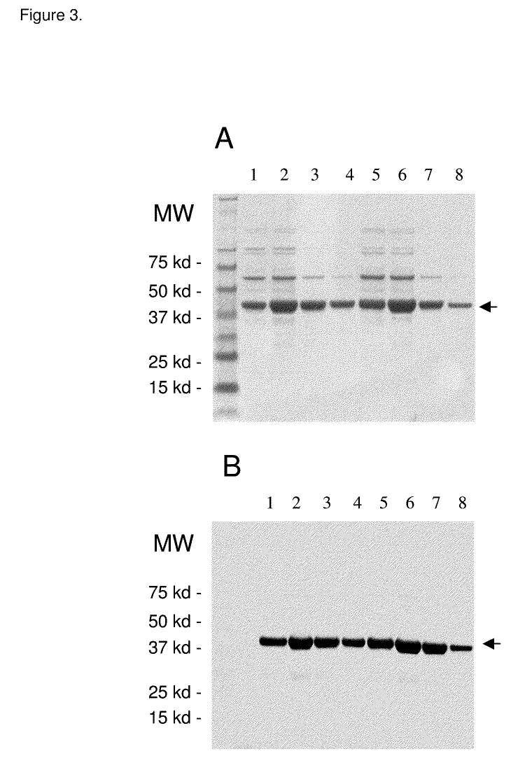

As discussed above, MGL is a serine hydrolase that converts monoacylglycerides (a.k.a. monoglycerides) to fatty acid and glycerol (Karlsson 1997). One potential fluorogenic substrate for MGL, 7-Hydroxycoumarin-arachidonate (7-HCA), was selected by substituting the glycerol moiety of 2-Arachidonate glycerol (2-AG) with a fluorescent probe such as a coumarin derivative. MGL catalyzes the hydrolysis of the non-fluorescent 7-HCA to produce arachidonic acid (AA) and a highly fluorescent 7-hydroxycoumarin (7-HC), having an excitation maximum at 355 nm, and emission maximum at 460 nm (FIG. 1). Hydrolysis of 7-HCA by MGL generated an assay window of more than 50-fold and thus was chosen as a substrate for studying MGL activity (FIG. 2).

MGL also catalyzed the hydrolysis of other ester-linked compounds such as 7-Hydroxycoumarinyl-γ-linolenate and 7-Hydroxycoumarinyl-6-heptenoate (FIG. 2 and Table 3). The stability of 7-HCA in assay buffer was monitored over time and found to be stable for at least 5 hours at 25° C. AAMCA, a fatty acid amide hydrolase (FAAH) substrate [Ramarao 2005], which has an amide-link between the arachidonate and coumarin moieties, was not a substrate for MGL (FIG. 2).

According to FIG. 2, each compound (10 μM) was individually incubated with 5 ng of MGL protein in assay buffer consisting of 50 mM Tris, 1 mM EDTA, pH 8, 10% DMSO in a total volume of 50 μl for 1 hour at 25° C. Increase in fluorescence at 460 nm was monitored continuously by a fluorometer. The fluorescence units were converted to the hydrolysis activity of MGL (pmol/min/mg protein). Data shown represents the mean±SD (n=3) from 2 individual experiments.

Table 3, shown below, depicts rates of hydrolysis for MGL with representative fluorogenic substrates. Rates were measured at reactions containing 3 ng/well MGL protein, a series of concentrations of above compounds in 10% DMSO, pH 8.0, and room temperature. The 7-HC fluorescence (λₑₓ=355 nm, λₑₘ=460 nm) was read after 10-minutes. The data were analyzed and the Vmax and Km calculated using the Michaelis-Menten equation. *ND means “not detectable activity.”

TABLE 3

| | Rate of hydrolysis | |
|---|---|---|
| | Vmax | |
| Substrate | (μmol/min/mg) | K_{M} (μM) |
| 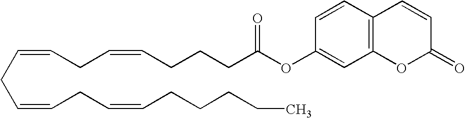 | 1.7 | 9.8 |
| 7-Hydroxycoumarinyl-arachidonate | | |
| 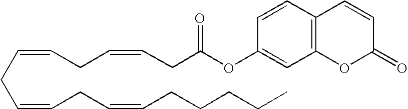 | 3.2 | 31 |
| 7-Hydroxycoumarinyl-γ-linolenate | | |
| 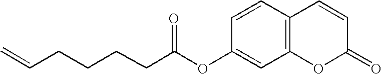 | 1.3 | 9.3 |
| 7-Hydroxycoumarinyl-6-heptenoate | | |
| 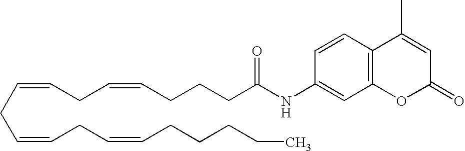 | ND* | ND* |
| 7-amino-4-methylcoumarin amide | | |

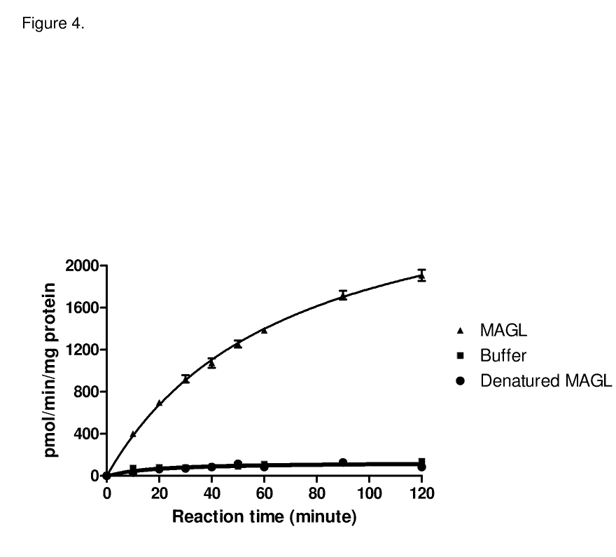

According to FIG. 4, 3 ng/well of MGL protein (triangle), or heat-denatured MGL protein (square), or buffer alone (circle) was incubated with 10 μM 7-HCA substrate in a total volume of 50 μl per well in 384 well plate at room temperature as described in the examples. Fluorescence was monitored over 2 hours using a Wallac 1420 Victor multilable HTS Counter. Data shown represents the mean±SD (n=3) from 2 individual experiments.

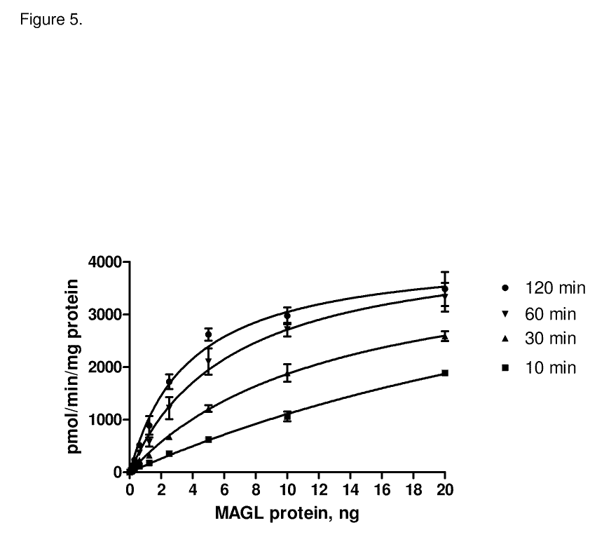

The effect of enzyme concentration on 7-HCA hydrolysis was investigated by incubating 10 μM 7-HCA with increasing amounts of MGL protein (FIG. 5). The amount of 7-HC produced was linear with enzyme concentrations up to 20 ng during a 10-minute reaction (FIG. 5). However, the hydrolysis rate of 7-HCA became less linear at longer time points. The duration of the linear production of 7-HC was dependent on enzyme concentration and was 30 minutes with 5 ng protein and 60 minutes with 3 ng protein per reaction (FIG. 5).

According to FIG. 5, 10 μM of 7-HCA substrate were incubated with the indicated amounts of MGL proteins at room temperature and 7-HC fluorescence was monitored at 10-, 30-, 60- or 120-minute points. Data shown is the hydrolysis activity of MGL (i.e., formation of 7-HC) (pmol/min/mg protein). Data shown represents the mean±SD (n=3) from 2 individual experiments.

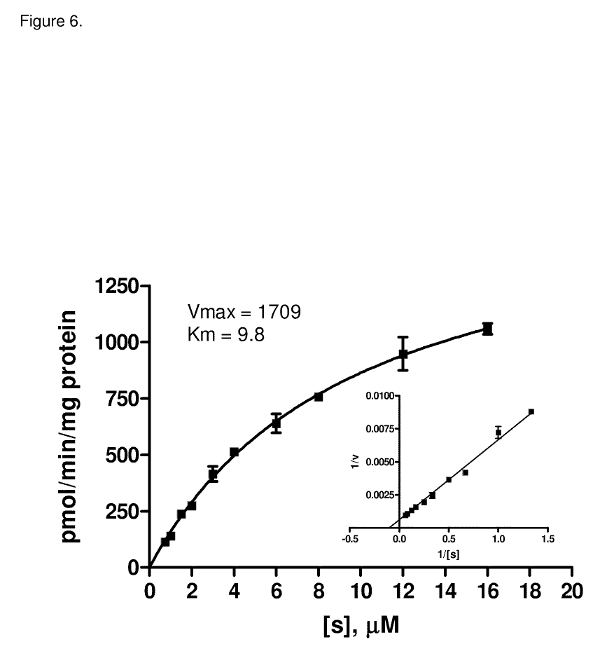

The kinetic constants, K_{M} (apparent) and Vₘₐₓ were calculated by determining the MGL activity with increasing substrate concentrations. 3 ng MGL protein were incubated with increasing 7-HCA concentrations, at pH 8, 25° C., for 10 minutes in a 384-well black plate, in a total volume of 50 μl. Specific activity of MGL was plotted against the substrate concentration and the data was fit to a non-linear regression equation (FIG. 6). 7-HCA hydrolysis followed apparent Michaelis-Menten kinetics, with an apparent Km of 9.8 μM and Vₘₐₓ of 1.7 μmoles min⁻¹ mg protein⁻¹. The Lineweaver-Burk plot is shown as an insert within FIG. 6.

According to FIG. 6, a 50 μl MGL reaction, containing 3 ng/well MGL protein, the indicated concentrations of 7-HCA and 10% DMSO, was performed at room temperature. The 7-HC fluorescence (λₑₓ=355 nm, λₑₘ=460 nm) was read after 10-minutes. The data were analyzed and the Vmax and K_{M} calculated using the Michaelis-Menten equation. A Lineweaver-Burk plot of 7-HCA hydrolysis by substrate is also shown.

The signal to noise ratio of the MGL fluorescence assay was determined over multiple experiments in a 384-well format. The Z-PRIME factor was found to be around 0.95, indicating that the assay is ideal for the screening of compound libraries.

### Inhibition of MGL Activity by Inhibitors

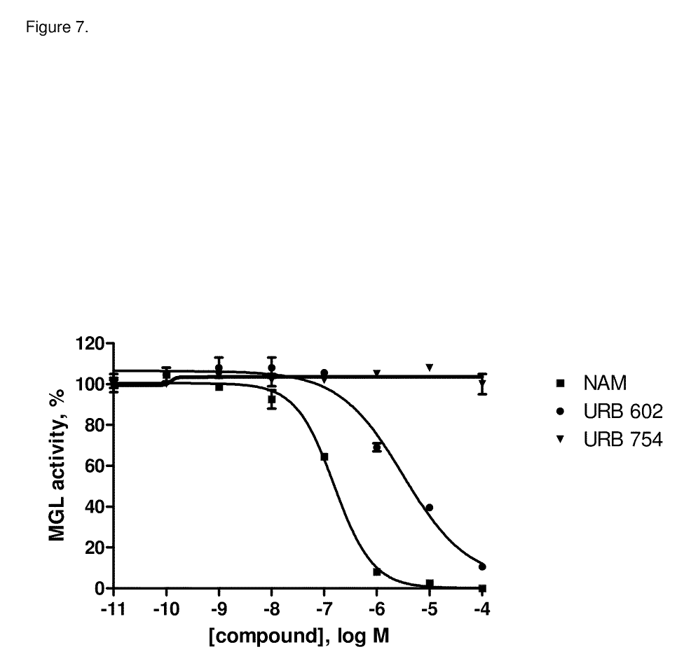

URB602 is a specific, irreversible, carbamate inhibitor of MGL activity (Makara et al., Nature Neurosci. 2005, 8(9): 1139-41). Inhibition of MGL activity by URB602 was studied by treating 3 ng of MGL protein with increasing concentrations of URB602 with 7-HCA as substrate at pH 8, and 25° C., for 60 minutes in a total volume of 50 μl. Under the conditions tested, URB602 inhibited MGL activity in a concentration-dependent manner, with an IC50 of 3.6 μM (FIG. 7). In contrast, N-Arachidonyl Maleimide (NAM), a potent irreversible inhibitor of MGL, exhibited an IC50 value of 155 nM. These results were comparable with the IC50 values reported with ³H-2-AG as substrate for both compounds (Makara 2005). URB754 was put through this MGL activity assay as a negative control, failing to inhibit MGL activity at concentrations up to 100 μM. It is important to note at this time that URB754 was previously reported to be a potent MGL inhibitor with IC50 values of 50 nM (Makara 2005) and later demonstrated to be false positive. The MGL inhibitory activity originally attributed to URB754 was later found to be caused by an unidentified impurity (Saario et al., Chem. Biol. 2006, 13(8):811-4, which is herein incorporated by reference.) Accordingly, URB754 did not inhibit MGL in this fluorescence assay and therefore serves as an important negative control.

According to FIG. 7, the assay was carried out by incubating 3 ng of MGL protein with 10 μM 7-HCA and indicated concentrations of compounds in 50 mM HEPES, pH 8, 1 mM EDTA and 10% DMSO, at 25° C. for 60 minutes. The percent inhibition of MGL activity was plotted against compound concentration, and the IC50 was calculated by fitting the curve to a non-linear regression equation using GRAPHPAD™ Prism 3.02. Each data point represents mean±SD (n=3) from at least 2 individual experiments.

### The Effect of Varying Assay Conditions on MGL Activity

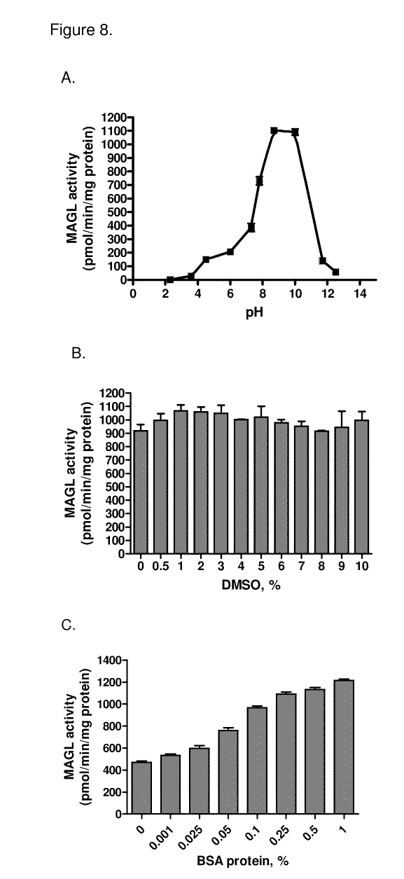

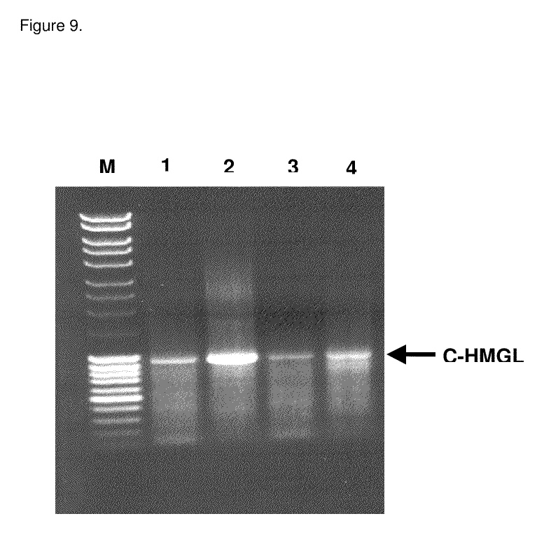

To determine the optimal pH range for the 7-HCA hydrolysis by MGL, the assay was performed at pH 2-12. The assays were conducted with 10 μM 7-HCA and 3 ng of MGL for 1 hour at 25° C. Results shown in FIG. 8a indicate that the activity peak of 7-HCA hydrolysis by MGL was seen at pH 8-10. Significant decreases in MGL activity were observed at buffer pH outside this range and therefore pH 8 was chosen for compound screening. Furthermore, extreme pH conditions could potentially affect the stability and solubility of compounds.

The effect of DMSO on MGL activity was analyzed by incubating 3 ng of MGL protein with 10 μM 7-HCA, with increasing DMSO concentrations in the assay. Results indicate that DMSO concentrations up to 10% of the final assay volume had no effect on MGL activity (FIG. 8b). While not wishing to be bound by any theory, the inclusion of DMSO in the assay is expected to increase the solubility of hydrophobic compounds.

The effect of BSA concentration on the assay was tested by treating 3 ng of the MGL protein with 10 μM 7-HCA in the presence of increasing amounts of BSA. MGL activity increased with increasing BSA concentration up to 1% (FIG. 8c). For compound screening in a 384-well format, a final BSA concentration of 0.1% was adopted as higher BSA concentration led to increased air bubbles in the wells which might be problematic, particularly when using automated liquid handling. 7-HCA is a highly lipophilic molecule, and the presence of BSA might prevent the non-specific binding of the substrate to the walls of the tubes, assist in the solubility of the substrate, improve the stability of the proteins, and to bind the product arachidonic acid to prevent product inhibition and thus drive the reaction forward.

According to FIG. 8, pH of the assay buffer (50 mM HEPES, 1 mM EDTA and 10% DMSO) was adjusted as indicated and the assay was performed by incubating 3 ng of MGL protein with 10 μM 7-HCA at 25° C. for 60 minutes. The effect of DMSO on hydrolysis of 7-HCA by MGL was tested by increasing the DMSO concentration in the assay as indicated. The effect of BSA on hydrolysis of 7-HCA by MGL was tested by increasing the final BSA concentrations as indicated. In all the experiments, each data point shown represents the mean±SD (n=3).

### Example 2

### E. Coli Expression System for Human MGL (HMGL)

### Materials

Oligonucleotides for HMGL gene synthesis were purchased from IDT Technologies (Coralville, Iowa). PCR enzymes were purchased from Stratagene (La Jolla, Calif.) and Takara-Mirus (Madison, Wis.). Restriction enzymes were purchased from New England Biolabs (Ipswich, Mass.). IPTG was purchased from Invitrogen (Carlsbad, Calif.). Chemically competent host strains E. coli DH5a, BL21 and TOP10 were purchased from Invitrogen. The pCOLD™ II expression vector was purchased from Takara-Mirus and pET16b (+) expression vector was purchased from Novagen (Madison, Wis.).

### HMGL Gene Synthesis

The HMGL gene was synthesized following the procedure as described (see PK Chanda et al., Protein Expr, Purif. 2006, 47(1):217-24, which is herein incorporated by reference). Briefly, in the gene assembly step equal volumes of each oligonucleotides at 40 μM concentration were mixed together and this mixture were further diluted 10-fold in 50 μl PCR reaction mixture containing appropriate DNA polymerase. The PCR reaction was carried out for 30 cycles. For gene amplification, the gene assembly reaction mixture (template) was diluted at least 10-fold in 50 μl complete PCR reaction mixture with the two outermost oligodeoxyribonucleotides at 1 μm concentration for 30 cycles. The PCR protocol for both gene assembly and gene amplification are essentially the same as described (Chanda 2006).

### Cloning and Sequencing

All PCR generated HMGL DNA fragments were agarose gel purified using a purification kit (Qiagen, Valencia, Calif.) and then ligated to either the PCR-blunt vector (using the Zero Blunt PCR cloning kit, Invitrogen) or other vectors of choice. The ligation reaction containing the purified HMGL DNA fragment and the vector of choice was carried out at 16° C. for 2-4 h and then used to transform TOP10 chemically competent E. coli cells according to the manufacturer's (Invitrogen) suggested protocol. The resulting clones were selected on LB agar plates containing 50 μg/ml of kanamycin (Teknova, Hollister, Calif.) for PCR-blunt vector or on appropriate antibiotic-containing agar plates for other cloning vectors. All clones were verified by DNA sequencing.

### Construction of Recombinant Expression Plasmids

The sequence verified HMGL DNA fragment was digested with NdeI and XhoI and then ligated either to pCOLD™ II or pET16b(+) expression vector (Novagen,) at the NdeI/XhoI sites. The ligation reaction was carried out at 16° C. for 4-6 h and then used to transform E. coli BL21 cells. The resulting clones were selected on LB agar plates containing 50 μg/ml of carbenicillin. The recombinant plasmid was verified by restriction digestion analyses followed by DNA sequencing.

### Expression of HMGL in BL21 E. Coli Cells

Colonies were picked from LB agar plates containing 50 μg/ml carbenicillin plates (Teknova) and were grown in LB medium containing the same antibiotic concentration as in the plate. Cells were grown at 37° C. with aeration until OD₆₀₀=0.5-0.7 was reached. The cell cultures were equilibrated at 16° C. for 20-30 min (for expression using pCOLD™ II vector) and then HMGL expression was induced by adding isopropyl β-D-galactoside (IPTG) to a final concentration of 1 mM at 16° C. for 3-72 hours. Expression of HMGL in pET16b (+) vector was carried out at 37° C. for 3-18 hours. 1 ml of the induced cultures were pelleted by centrifugation (14,000 g×5 min) and total cell proteins were analyzed by NuPAGE 4-20% Bis-Tris gradient SDS-PAGE (Invitrogen). Protein bands were visualized by staining with SIMPLYBLUE™ SafeStain solution (Invitrogen) followed by destaining with distilled water.

### Protein Purification

Cells (50 ml) containing the appropriate HMGL recombinant plasmids were induced with IPTG as herein described in the Materials subsection of this example. Cells were harvested from liquid culture by centrifugation at 5,000×g for 10 min followed by 1×PBS buffer wash. Cells were then resuspended in 3.0 ml of BugBuster reagent (Novagen) and 10 μl Lysonase Bioproceesing Reagent (Novagen) was added for efficient protein extraction. The cell suspension was incubated on a shaking platform at a slow setting for 10-20 min at room temperature. Insoluble debris were removed by centrifugation at 16,000×g for 20 min at 4° C. Soluble extract was then loaded onto pre-charged His-Bind column (Novagen) and protein was purified following manufacturer's suggested protocol.

### HMGL Assay

The assays were performed in 96-well black plates (Nalge Nunc International, Rochester, N.Y.) in a total volume of 100 μl per well. The assay buffer consists of 10 mM Tris-HCl, pH 7.2, 1 mM EDTA and 0.1% BSA. The assay was initiated by adding 8 ng of purified protein samples followed by the addition of 1 μm substrate, 7-HCA, to each well. The plates were incubated at room temperature (˜25° C.) for 1 h and then read on a Flex Station plate reader at λₑₓ=355 nM and λₑₘ=460 nM.

### Recombinant Plasmids

The coding sequences of both codon-optimized (SEQ ID NO:1) and non-optimized HMGL cDNA (SEQ ID NO:2) and the corresponding oligonucleotides required for PCR-directed HMGL gene synthesis (SEQ ID NOs:3-98) were generated using a web-based DNA codon optimization algorithm (UPGENE™; see Gao et al., 2004, which is incorporated herein by reference). The synthesis of codon-optimized human MGL (“C-HMGL”) cDNA and non-codon-optimized human MGL (“N-HMGL”) cDNA by PCR-directed gene synthesis method (U.S. Provisional Application No. 60/898,448, which is herein incorporated by reference) is shown in FIG. 9. SEQ ID NO:3-26 represent C-HMGL sense strand oligonucleotides number 1 through 24, respectively. SEQ ID NO:27-50 represent the counterpart C-HMGL antisense strand oligonucleotides number 1 through 24, respectively. SEQ ID NO:51-74 represent N-HMGL sense strand oligonucleotides number 1 through 24, respectively. SEQ ID NO:75-98 represent the counterpart N-HMGL antisense strand oligonucleotides number 1 through 24, respectively. The C-HMGL cDNA PCR product was digested with NdeI/XhoI, and then purified from agarose gel using Qiaquick gel extraction kit (Qiagen, CA). They were cloned either into pCOLD™ II or pET16b(+) vector at the NdeI/XhoI sites. The resulting recombinant HMGL constructs are capable of producing either six or ten histidine tag (H6 or H10) at the NH2-terimus of the protein. The pCOLD™ II expression vector contains a cold shock promoter (cspA) that is highly active at lower temperature (16° C.). This vector also contains translational enhancer (TEE) sequence (SEQ ID NO:101), mutated ribosome binding site (RBS) (GAGG) and 6Xhis-tag (H6) for protein purification. The presence of TEE sequence in conjunction with the mutated RBS is intended for high-level expression of cloned genes from this vector. However, the pET16b (+) expression vector contains a powerful T7 promoter with decahistidine-tag (H₁₀) at the NH2 terminus to produce NH2-terminus his-tagged fusion protein.

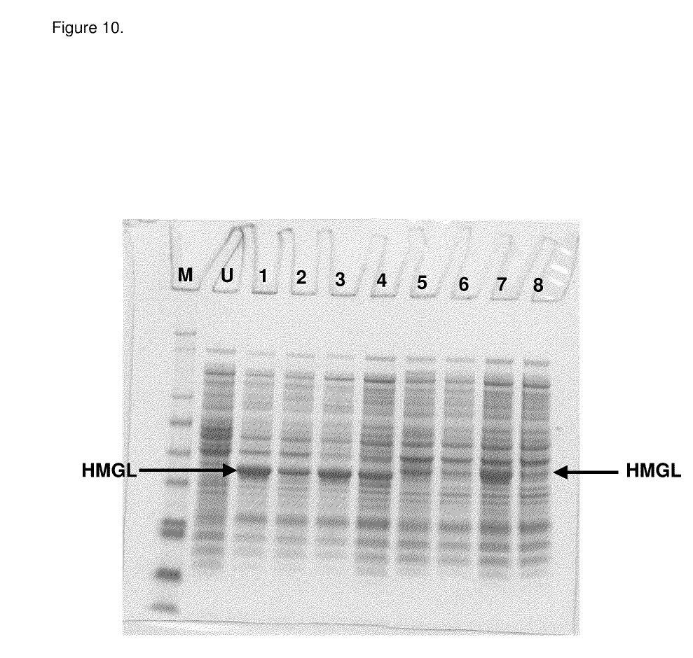

According to FIG. 9, 5 μl of mixed oligonucleotides (FIG. 10) at 40 μM concentration were used for gene assembly and gene synthesis as described in Example 2. Lane M, Mass Ruler DNA ladder (Fermentas, Hanover, Md.) molecular weight markers: Lane1, C-HMGL gene synthesis using PfuUltra High Fidelity DNA polymerase: lane 2, C-HMGL synthesis with PICOMAXX™ High Fidelity DNA polymerase: lane 3, C-HMGL synthesis with PfuTURBO™ Hotstart DNA polymerase: lane 4, C-HMGL synthesis with HERCULASE™ Hotstart DNA polymerase.

### Expression of Codon-Optimized Human MGL (“C-HMGL”) and Non-Optimized Human MGL (“N-HMGL”)

High levels expression of both C-HMGL and N-HMGL could be seen in E. coli BL21 cells upon induction with IPTG (FIG. 10). However, expression levels were found to be higher with codon optimized MGL sequence as compared to the non-codon optimized sequence (FIG. 10). This pattern was observed when expression was carried out in either LB or TB medium.

According to FIG. 10, C-HMGL and N-HMGL sequences were cloned either in pCOLD™ II or pET16b (+) expression vectors and were expressed in E. coli BL21 cells following IPTG induction for 4 h at either 16° C. or 37° C. as described in Materials and Methods. Lane M, MassRuler molecular weight markers; lane U, whole cell lysates of uninduced E. coli cells; lane 1, expression of C-HMGL from pCOLD™ II vector in LB medium; lane 2, expression of N-HMGL from pCOLD™ II vector in LB medium; lane 3, expression of C-HMGL from pCOLD™ II vector in TB medium; lane 4, expression of N-HMGL from pCOLD™ II vector in TB medium; lane 5, expression of C-HMGL from pET16b (+) vector in LB medium; lane 6, expression of N-HMGL from pEt16b (+) vector in LB medium; lane 7, expression of C-HMGL from pET16b (+) vector in TB medium; lane 8, expression of N-HMGL from pET16b (+) vector in TB medium.

### Effect of IPTG Concentration on HMGL Expression

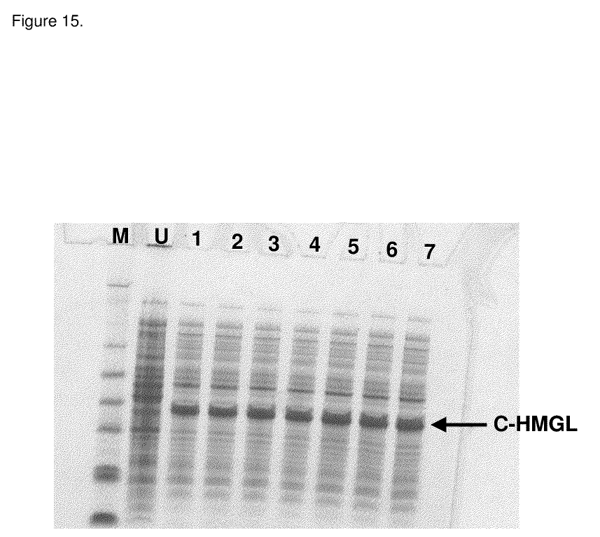

Both C-HML and N-HMGL were expressed in presence of varying concentrations of IPTG. Similar levels of HMGL expression were obtained using the IPTG concentration ranging between 0.1 mM to 2.0 mM (FIGS. 15-18).

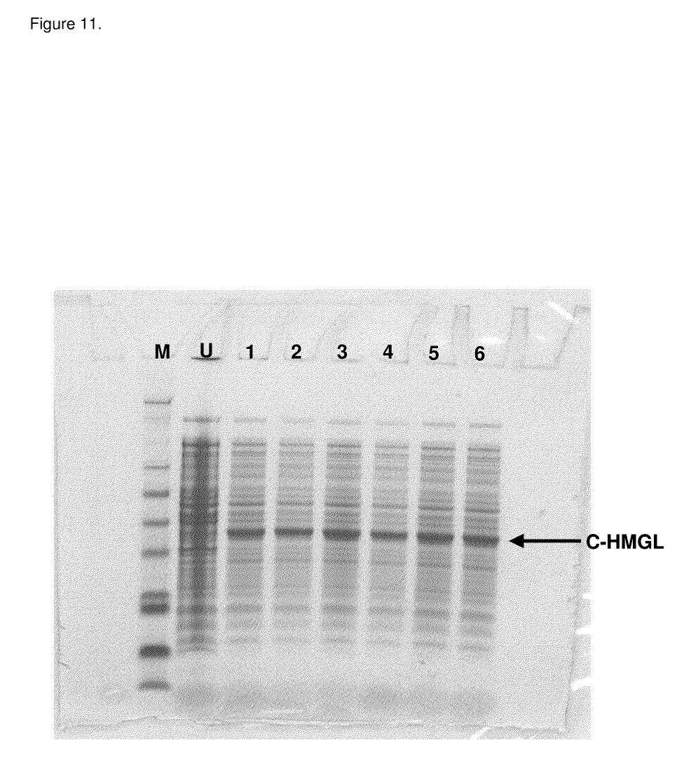

According to FIG. 11, the C-HMGL sequence was expressed from pCOLD™ II vector in E. coli BL21 cells at 16° C. for 16 hours with varying concentrations of IPTG. Total cell protein samples were separated on 4-20% Bis-Tris SDS-PAGE under reducing conditions and stained with SimplyBlue SafeStain solution (Invitrogen). Lane M, MassRuler molecular weight markers; lane U, whole cell lysates of uninduced E. coli cells; lane 1, C-HMGL induction with 0.1 mM IPTG; lane 2, C-HMGL induction with 0.2 mM IPTG; lane 3, C-HMGL induction with 0.5 mM IPTG; lane 4, C-HMGL induction with 1.0 mM IPTG; lane 5, C-HMGL induction with 1.5 mM IPTG; lane 6, C-HMGL induction with 2.0 mM IPTG.

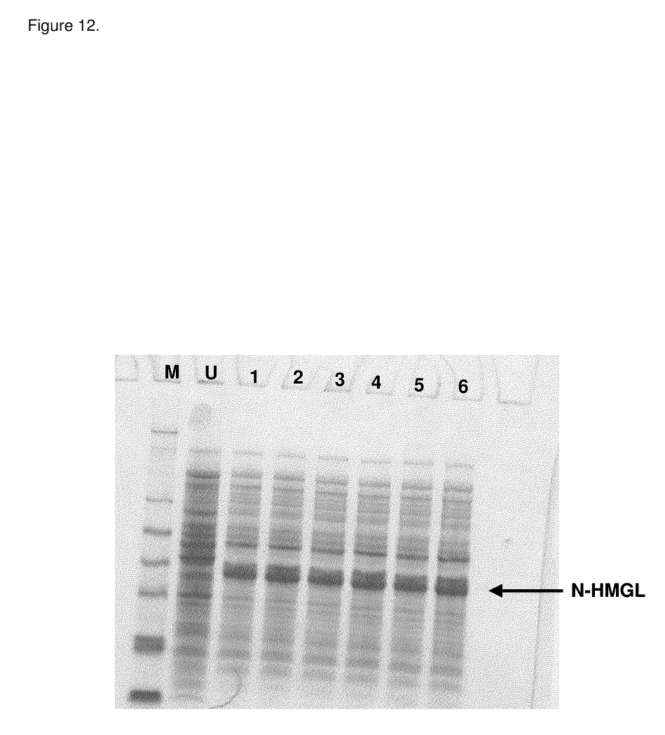

According to FIG. 12, the N-HMGL sequence was expressed from pCOLD™ II vector in E. coli BL21 cells at 16° C. for 16 hours with varying concentrations of IPTG. Total cell protein samples were separated on 4-20% Bis-Tris SDS-PAGE under reducing conditions and stained with SIMPLYBLUE™ SafeStain solution (Invitrogen). Lane M, MassRuler molecular weight markers; lane U, whole cell lysates of uninduced E. coli cells; lane 1, N-HMGL induction with 0.1 mM IPTG; lane 2, N-HMGL induction with 0.2 mM IPTG; lane 3, N-HMGL induction with 0.5 mM IPTG; lane 4, N-HMGL induction with 1.0 mM IPTG; lane 5, N-HMGL induction with 1.5 mM IPTG; lane 6, N-HMGL induction with 2.0 mM IPTG.

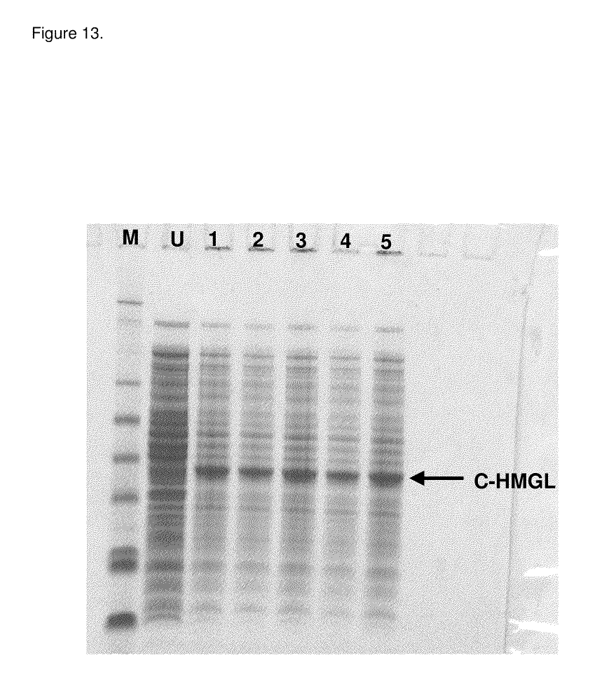

According to FIG. 13, the C-HMGL sequence was expressed from pET16b (+) vector in E. coli BL21 cells at 37° C. for 4 hours with varying concentrations of IPTG. Total cell protein samples were separated on 4-20% Bis-Tris SDS-PAGE under reducing conditions and stained with SIMPLYBLUE™ SafeStain solution (Invitrogen). Lane M, MassRuler molecular weight markers; lane U, whole cell lysates of uninduced E. coli cells; lane 1, C-HMGL induction with 0.1 mM IPTG; lane 2, C-HMGL induction with 0.2 mM IPTG; lane 3, C-HMGL induction with 0.5 mM IPTG; lane 4, C-HMGL induction with 1.0 mM IPTG; lane 5, C-HMGL induction with 2.0 mM IPTG.

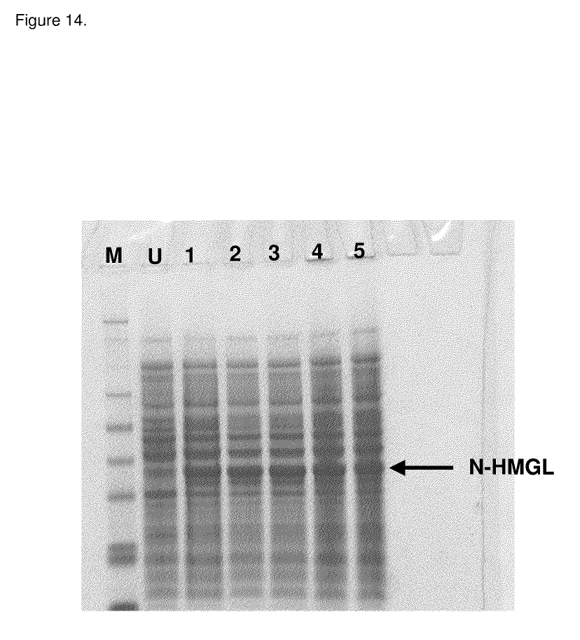

According to FIG. 14, the N-HMGL sequence was expressed from pET16b (+) vector in E. coli BL21 cells at 37° C. for 4 hours with varying concentrations of IPTG. Total cell protein samples were separated on 4-20% Bis-Tris SDS-PAGE under reducing conditions and stained with SIMPLYBLUE™ SafeStain solution (Invitrogen). Lane M, MassRuler molecular weight markers; lane U, whole cell lysates of uninduced E. coli cells; lane 1, N-HMGL induction with 0.1 mM IPTG; lane 2, N-HMGL induction with 0.2 mM IPTG; lane 3, N-HMGL induction with 0.5 mM IPTG; lane 4, N-HMGL induction with 1.0 mM IPTG; lane 5, N-HMGL induction with 2.0 mM IPTG.

### Effect of Post-Induction Time on HMGL Expression

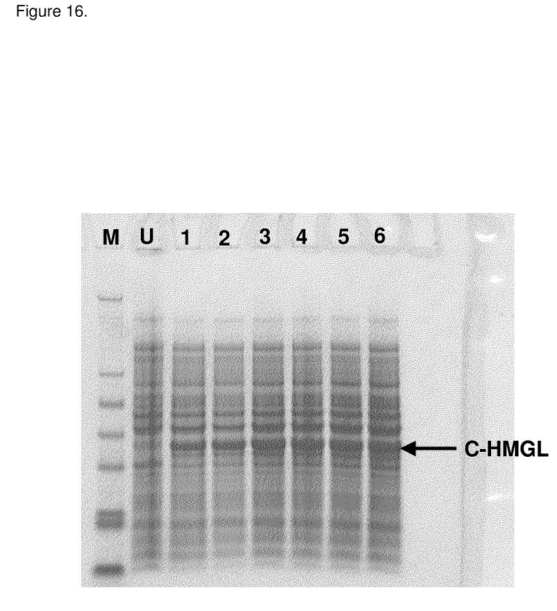

Similar levels of HMGL expression were obtained when the expression was carried out using pCOLD™ II vector between 4-72 h post IPTG induction at 16° C. (FIG. 15) and 3-8 h post IPTG induction using pET 16b (+) vector at 37° C. (FIG. 16). It is interesting to point out that the synthesis of HMGL was still maintained even at 72 h after cold shock indicating that the cells with the pCOLD™ II vector system retained the protein-synthesizing capability for more than 2 days at 16° C.

According to FIG. 15, the C-HMGL cDNA was cloned in pCOLD™ II vector and expression was carried out in E. coli BL21 cells at 16° C. by inducing with 0.5 mM IPTG for various time points. Cells were harvested and analyzed by SDS-PAGE. Lane M, MassRuler DNA ladder molecular weight markers; lane U, whole cell lysates of uninduced E. coli cells; lane 1, 3 h IPTG induction; lane 2, 6 h IPTG induction; lane 3, 12 h IPTG induction; lane 4, 18 h IPTG induction; lane 5, 24 h IPTG induction; lane 6, 48 h IPTG induction; lane 7, 72 h IPTG induction.

According to FIG. 16, the C-HMGL cDNA was cloned in pET16b(+) vector and expression was carried out in E. coli BL21 cells by inducing with 0.5 mM IPTG for various time points at 37° C. Cells were harvested and analyzed by SDS-PAGE. Lane M, MassRuler DNA ladder molecular weight markers; lane U, whole cell lysates of uninduced E. coli cells; lane 1, 3 h IPTG induction; lane 2, 6 h IPTG induction; lane 3, 9 h IPTG induction; lane 4, 12 h IPTG induction; lane 5, 18 h IPTG induction; lane 6, 24 h IPTG induction.

### Protein Purification

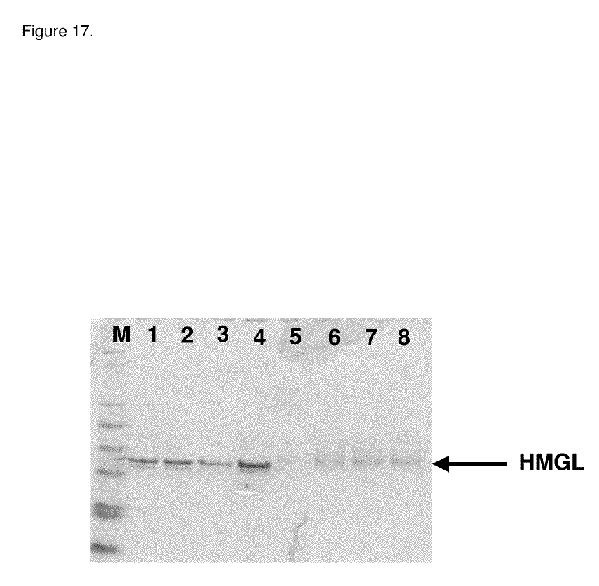

HMGL proteins expressed from the expression vectors used in this study were purified using His-Bond resins. The yield of soluble protein was in the range of 10-15 mg/L from pCOLD™ II vector and 3-5 mg/L from pET16b (+) vector (FIG. 17).

According to FIG. 17, the HMGL proteins expressed in E. coli BL 21 cells using either LB or TB medium from various expression constructs were purified by Ni-IDT His-Bond columns as described in the examples. The gel was set up as follows: Lane M, MassRuler DNA ladder molecular weight markers; lane 1, C-HMGL pCOLD™ II (LB); lane 2, N-HMGL/pCOLD™ II (LB); lane 3, C-HMGL/pCOLD™ II (TB); lane 4, N-HMGL/pCOLD™ II (TB); Lane 5, C-HMGL/pET16b (+) (LB); lane 6, N-HMGL/pET16b (+) (LB); Lane 7, C-HMGL/pET16b (+) (TB); lane 8, N-HMGL/pET16b (+) (TB).

### Functional Activity of HMGL

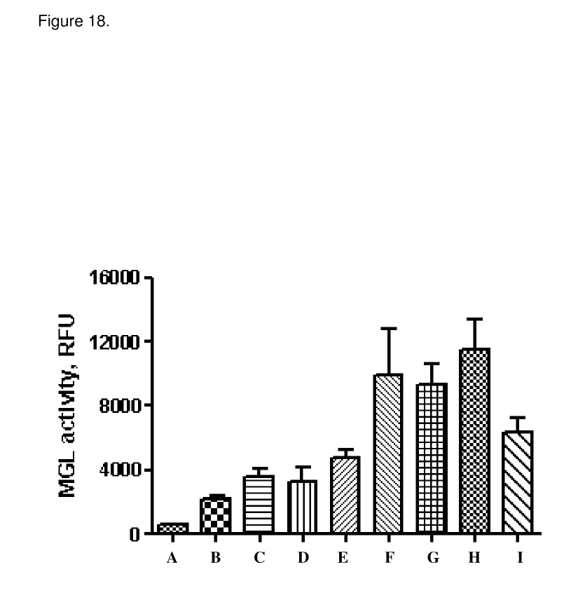

Functional activity of HMGL was determined using the fluorogenic substrate 7-HCA as herein described. The HMGL protein was found to be highly functional. However, HMGL protein produced from pET16 b (+) construct at 37° C. had a consistently and unexpectedly 3-5 fold higher specific activity than that produced from pCOLD™ II vector (FIG. 18).

According to FIG. 18, the HMGL protein expression was carried out in E. coli BL21 cells using either LB or TB medium. The corresponding proteins were purified using His-Bond columns and analyzed for monoglycerol lipase activity as described previously. The different constructs used for assay are denoted as follows. A, buffer alone: B, C-HMGL/pCOLD™ II (LB), C, N-HMGL/pCOLD™ II (LB), D, C-HMGL/pCOLD™ II (TB), E, N-HMGL/pCOLD™ II (TB); F, C-HMGL/pET16b (+) (LB); G, N-HMGL/pET16b (+) (LB); H, C-HMGL/pET16b (+) (TB); I, N-HMGL/pET16b (TB).

What is claimed is:

1. A method for identifying an agent that affects an activity of a monoglycerol lipase (“MGL”), the method comprising (a) combining (i) a test compound, (ii) an MGL and (iii) a fluorogenic substrate to form an assay mixture; and (b) detecting fluorescence emission of the assay mixture; wherein a change in the fluorescence emission compared to the fluorescence detected without adding the test compound indicates that the test compound is an agent that affects an activity of an MGL.

2. The method of claim 1, wherein the MGL is a recombinant MGL.

3. The method of claim 2, wherein the MGL is expressed in bacteria.

4. The method of claim 3, wherein the MGL is expressed in E. coli.

5. The method of claim 1, wherein the MGL comprises an amino acid sequence that is at least 37% identical to SEQ ID NO:99.

6. The method of claim 5, wherein the MGL is encoded by a polynucleotide comprising a nucleotide sequence of SEQ ID NO:1.

7. The method of claim 1, wherein the fluorescent substrate comprises a fatty acyl part joined to a fluorescent moiety via an ester linkage.

8. The method of claim 1, wherein the assay mixture is incubated for 60 minutes at 25° C. at pH 8 prior to detecting fluorescence emission.

9. The method of claim 1, wherein the activity of the MGL in the presence of the agent is decreased at least 30% relative to the activity of MGL determined in the absence of the agent.

10. The method of claim 9, wherein the agent is capable of increasing the level of an endocannabinoid in a physiological system.

11. An isolated polynucleotide comprising a sequence set forth in SEQ ID NO:1.

12. A recombinant monoglycerol lipase (“MGL”) polypeptide produced by (a) inserting the polynucleotide of claim 11 into a bacterial expression vector; and (b) expressing the polypeptide in a bacterial host cell.

13. The recombinant MGL polypeptide of claim 12, wherein the bacterial expression vector is a pCOLD™ II vector.

14. The recombinant MGL polypeptide of claim 12, wherein the bacterial expression vector is a pET™16b(+) vector.

15. The recombinant MGL polypeptide of claim 14, wherein the MGL has an about 3 fold to about 5 fold higher specific activity relative to the specific activity of a recombinant MGL polypeptide of claim 13.

16. A kit comprising a monoglycerol lipase (“MGL”), instructions and packaging materials.

17. The kit of claim 16, wherein the MGL is a recombinant MGL polypeptide of claim 12.

18. The kit of claim 17, further comprising a fluorogenic substrate.

19. The kit of claim 18 wherein the fluorogenic substrate is a 7-hydroxycoumarinyl-arachidonate.

20. The kit of claim 19, further comprising a buffer comprising 50 mM 4-(2-hydroxyethyl)-1-piperazineethanesulfonic acid and having a pH of about 8.0.
